(19) United States
(12) Patent Application Publication
(10) Pub. No.: US 2004/0034209 A1
(43) Pub. Date: Feb. 19, 2004

(54) Vaccination of hiv infected persons following highly active antiretrovial therapy

(76) Inventors: David Ho, New York, NY (US); Martin Markowitz, New York, NY (US); MICHEL KLEIN, LYON CEDEX (FR); RAPHAELLE EL HABIB, LYON CEDEX (FR)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/182,067

(22) PCT Filed: Jan. 26, 2001
(86) PCT No.: PCT/US01/02766

(51) Int. Cl. C07H 21/04; A61K 31/70; A01N 43/04; A61K 39/21; C12N 15/00; C12N 15/09; C12N 15/63; C12N 15/70; C12N 15/74

(52) U.S. Cl. 536/23.72; 435/320.1; 424/208.1

(57) ABSTRACT

The present invention provides a method of permitting cessation of antiviral therapy on HIV-infected subjects without virus rebound or with at least a delayed virus rebound or a decreased post rebound set-point. The method comprises the re-induction of HIV-specific immune responses using a vaccination strategy to induce both humoral and cell-mediated immunity. The present invention achieves an immunological control of persistent infectious virus after discontinuation of antiviral therapy. The vaccine strategy according to the invention is both safe and immunogenic in the subject HIV-infected patient population.

### BACKGROUND OF THE INVENTION

1. Field of the Invention

This invention relates to the field of methods of treating HIV-infected patients.

2. Summary of the Related Art

HIV infection is characterized by high levels of virus replication at all stages of infection. Virus replication causes increased levels of CD4 cell destruction and turnover, and when unchecked, immunodeficiency, AIDS and death. This model of pathogenesis has prompted a dramatic change in the treatment paradigm which has evolved from late intervention in symptomatic individuals to a “hit early, hit hard” strategy.

Perelson and co-workers developed a mathematical model based on the biphasic decay of plasma HIV RNA after initiating potent antiviral therapy. The model hypothesized that two to three years of treatment with a completely suppressive regimen could result in a virologic remission or “eradication of infection” in HIV-infected individuals. The two to three year estimate required complete suppression of virus replication, the absence of any additional slower decaying compartments and/or the absence of sequestered areas of virus replication.

Subsequently, it has been demonstrated that a pool of latently infected resting CD4+ T-cells harboring infectious provirus persists in individuals treated with highly active antiretroviral therapy (HAART). The decay characteristics of this compartment remain somewhat controversial. Finzi and coworkers have performed longitudinal quantitative HIV-1 co-culture studies on HAART treated subjects. They have concluded that this pool decays with an average half-life of 44 months. Studies by Zhang et al and Ramratnam et al suggest that the inherent decay rate of the latent pool is much shorter and is approximately 6 months on average. Given these decay rates, eradication with antiviral therapy alone would require a minimum of 10 years of complete suppression of viral replication.

Ramratnam and co-workers demonstrated that in individuals exhibiting prolonged decay characteristics of the latent pool, ongoing virus replication was evident. Other investigators have come to similar conclusions by measuring markers of ongoing replication including HIV-1 mRNA species in PBMC and levels of 2LTR circles in PBMC. As would be predicted, attempts to discontinue therapy in apparently well-suppressed individuals have been associated with virologic rebound within days to weeks of therapy discontinuation. Furthermore, it has been observed that the initial rate at which the plasma viremia increases (doubling time) is somewhat uniform and generally observed to be approximately 1.5 days.

The use of combination antiretroviral therapy has markedly altered the natural history of HIV-1 infection. Both HIV-1-related mortality and morbidity have been significantly reduced by the introduction of combination antiretroviral therapies including potent inhibitors of HIV protease and reverse transcriptase [Palella, 1998]. Despite these gains, however, it is clear that these therapies are less than ideal. Long term antiretroviral therapy is associated with significant toxicities, both short term and long term [Carr, 1998; Carr, 1998; Sulkowski, 2000; Vigouroux, 1999; Brinkman, 1999; Echevarria, 1999]. Perhaps most disturbing are the metabolic consequences of long term therapy. Syndromes including hyperlipidemias with the potential for accelerated atherosclerosis, disfiguring peripheral fat and muscle wasting and central fat deposition, as well as hyperglycemia and glucose intolerance has been associated with long term antiviral therapy. Furthermore, it is clear that the current therapies require a degree of patient adherence that is often difficult to achieve. The result of non-adherence is treatment failure and may allow for the emergence of drug resistant viruses. Therefore, treatment strategies designed to limit the duration of antiviral therapy are clearly desirable.

### SUMMARY OF THE INVENTION

The present invention provides a method of permitting cessation of antiviral therapy on such HIV-infected subjects without virus rebound, with a delayed viral rebound, or with decreased post-rebound set point. The method comprises the re-induction of HIV-specific immune responses using a vaccination strategy to induce both humoral and cell-mediated immunity. The present invention achieves an immunological control of persistent infectious virus after discontinuation of antiviral therapy. The vaccine strategy according to the invention is safe and induces immune responses in the HIV-infected patient population.

The present invention is directed to a method of stimulating efficient CD4+ and CD8+ responses in a human infected with an HIV retrovirus who has a viral load of less than 10,000, preferably less than 5,000, viral copies per ml of plasma and a CD4+ T-cell count of above 300 cells/ml, preferably above 500 cells/ml, and who has been treated with a potent combination of antiviral agents that contributed to a lower viral copy number and equal or higher CD4+ cell count than before treatment. The method comprises administering a nucleic acid-based vaccine that enters the cells and intracellularly produces HIV-specific immunogens for presentation on the cell's MHC class I and MHC class II molecules in an amount sufficient to stimulate HIV-specific CD4+ and CD8+ T-cell responses, thereby reversing the otherwise observed population decline of these cells during antiretroviral therapy. In a preferred embodiment, the human has been treated with HAART therapy that resulted in the human having a viral load of less than 1,000 viral copies per ml of blood serum and a CD4+ cell count of above 500 cells/ml.

The method employs a vaccine that is a nucleic acid-based vaccine comprising naked or vectored nucleic acid. According to a preferred embodiment, the vaccine comprises an attenuated recombinant poxvirus, particularly NYVAC or ALVAC, that includes one or more nucleic acids encoding more or more HIV-specific immunogens. The vaccine optionally further comprise an adjuvant and is administered one or multiple times. The vaccine is optionally combined with an HIV antigen as well as immunostimulatory or co-stimulatory molecules such as interleukin 2 or CD40 ligand, respectively, in an amount that is sufficient to potentiate T-cell responses, in particular CD8+ responses.

The method of the invention is particularly useful for people who have been infected by HIV and who have demonstrated CD4+ and/or CD8+ T cell responses to HIV antigens, such as people who have demonstrated proliferative T-cell responses to gp120 envelope protein or p24 or both gp120 envelope and p24 Gag antigen. But the method of the invention is also useful for people who have lost their CD4+ and/or CD8+ T cell responses to HIV antigens, such as people who have lost their proliferative T cell response to gp120 or p24.

### BRIEF DESCRIPTION OF THE DRAWINGS

FIG. 1 displays plasma RNA and CD4+ T-cell levels for HIV-infected patients undergoing HAART.

FIG. 2 is a bar graph displaying the number of HIV-infected subjects undergoing HAART having plasma HIV RNA levels of less than 200, 50, and 25 copies/ml.

FIGS. 3A and 3B display CTLp frequencies for two patients undergoing HAART.

FIGS. 4A-4D display the percent of CD8+ IFN-secreting cells to specific HIV antigens for four HAART patients receiving HIV vaccination according to the invention.

FIGS. 5A-5D display plasma viremia in four HAART patients receiving HIV vaccination according to the invention.

FIGS. 6A-6F display plasma HIV RNA and CD4 T-cell count levels as a function of days on therapy for several patients.

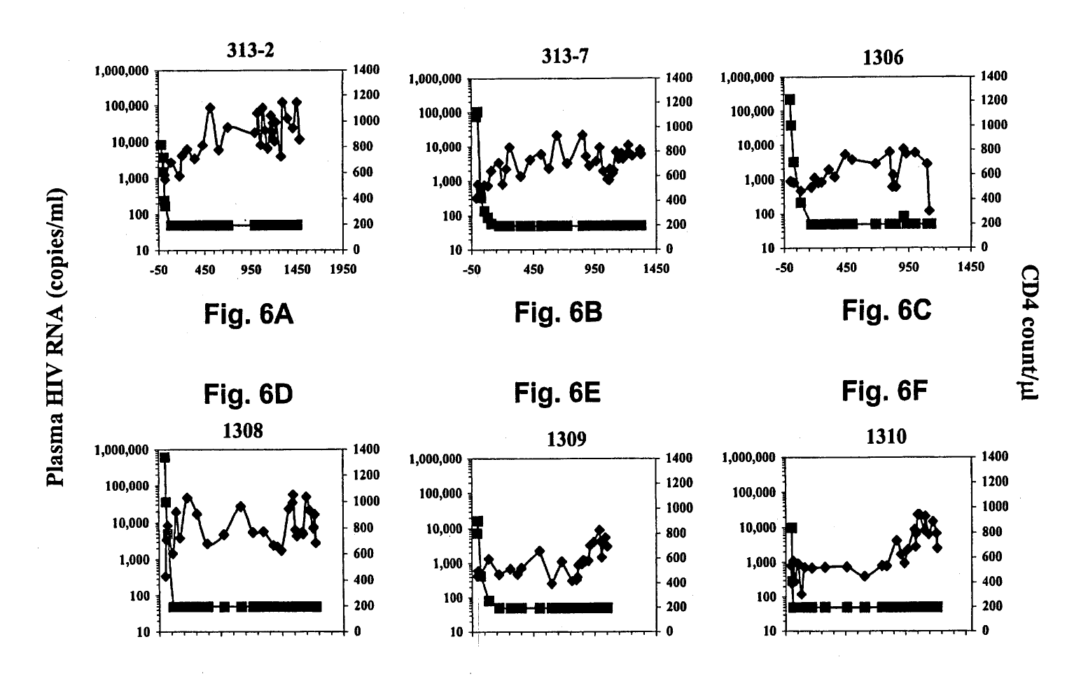

FIGS. 7A-7F display anti-gp120 and anti-p24 antibody titers for several patients as a function of days post vaccination.

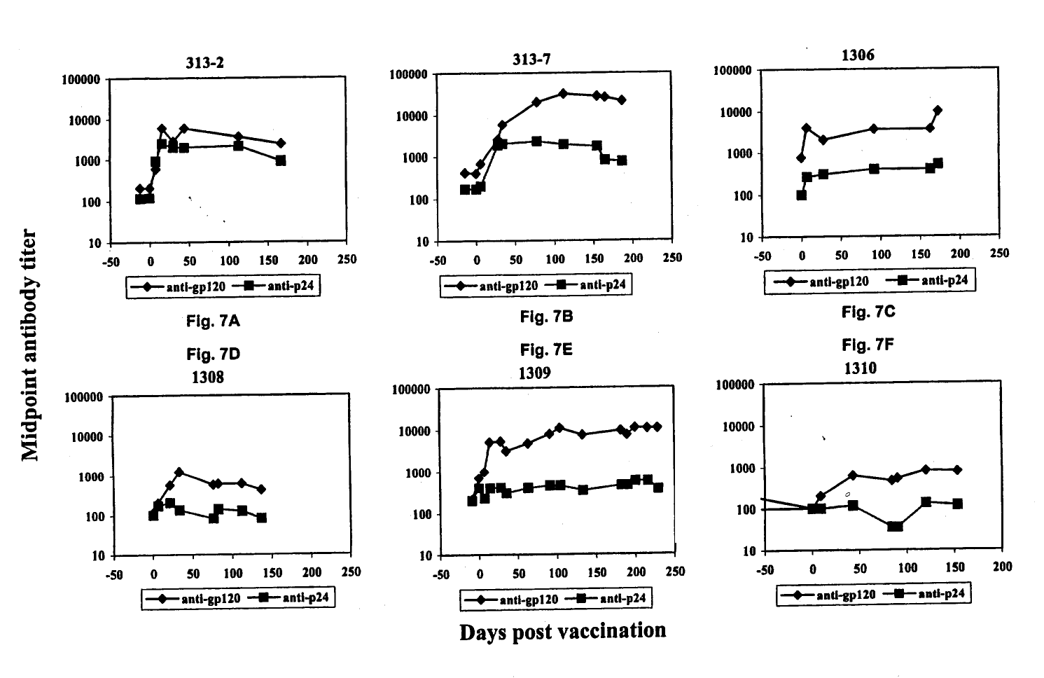

FIGS. 8A-8F displays intracellular cytokine staining.

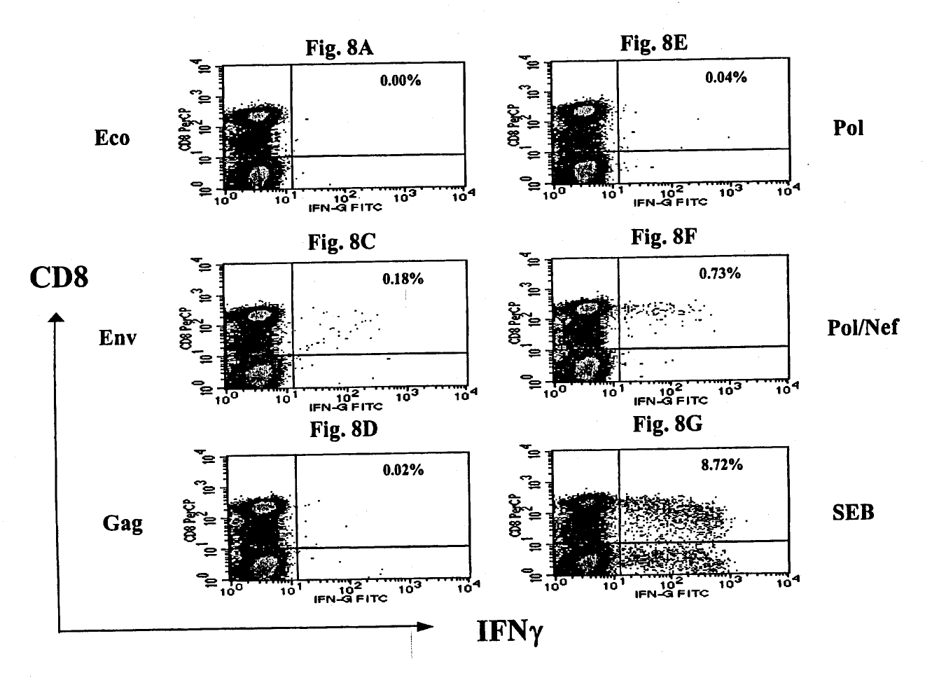

FIG. 9A-9F display data relating to various HIV antigens.

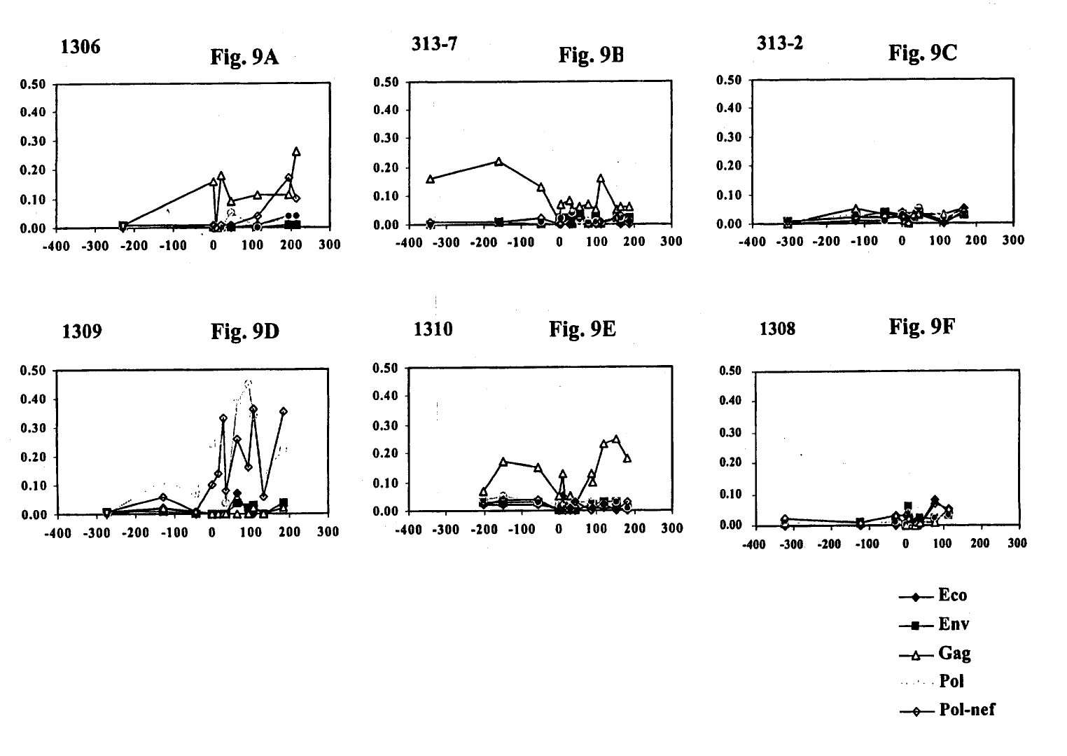

FIGS. 10A-10F display stimulation indexes as a function of days post vaccination.

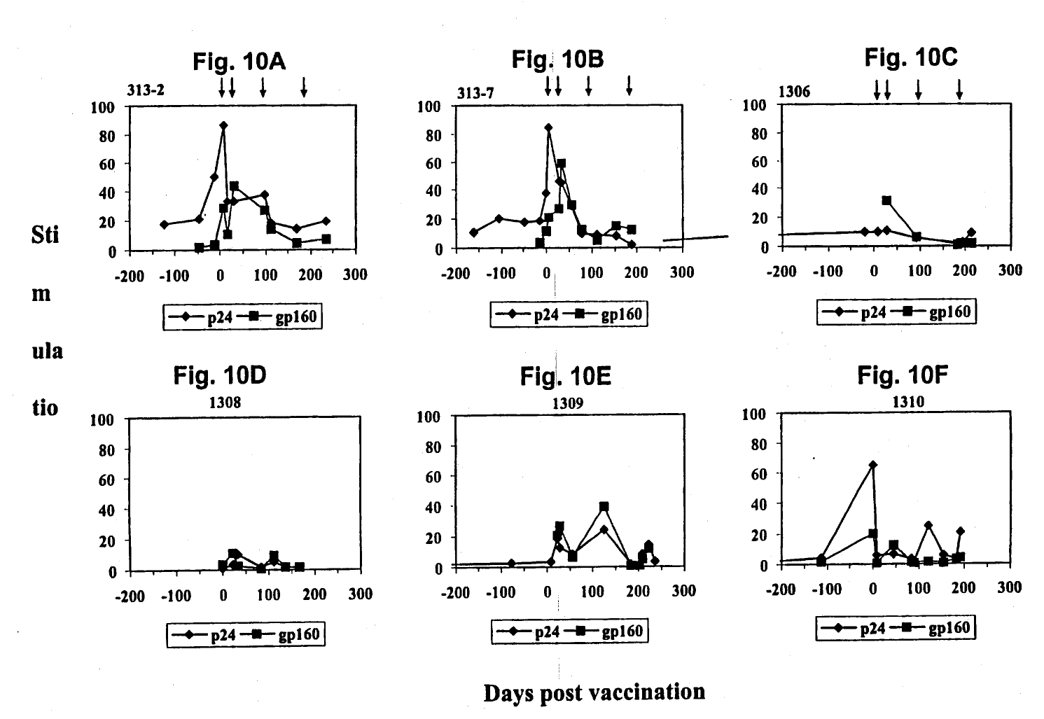

FIGS. 11A-11-F display stimulation indexes as a function of days post vaccination.

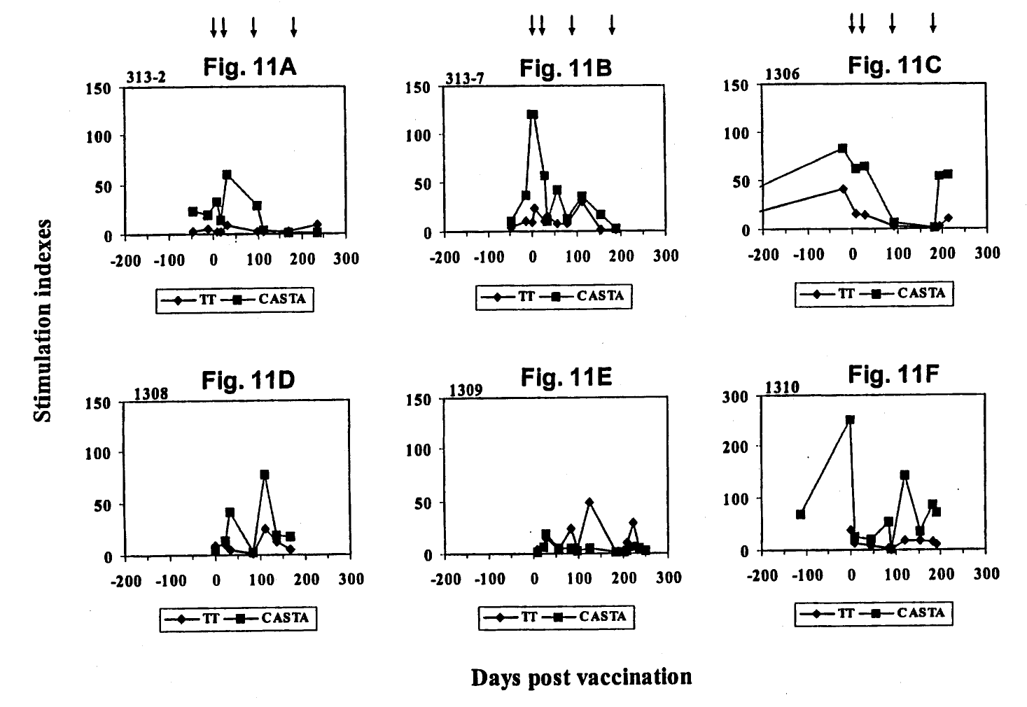

### DETAILED DESCRIPTION OF THE INVENTION

The present invention provides a novel therapeutic modality for treating persons infected with a lymphotropic or immune-destroying retroviral infection. Today, a physician presented with a patient whose immune system is compromised by retroviral infection can select to treat that patient with a host of powerful antiviral agents, including inhibitors of viral proteases and reverse transcriptase. This is known as highly active anti-retroviral therapy (HAART). The conventional HAART protocols are complex and difficult for patients to follow. The drugs also have a number of problematic side effects. In addition, these expensive and complicated treatments fail to eliminate the virus; they merely hold the virus in check. If the patient is non-compliant, the viral count rebounds. Accordingly, for the vast majority of patients, a lifetime of drugs is advised.

The present invention comprises the discovery that after HIV infection, HAART treatment that decreases the viral load can be discontinued using an anti-HIV vaccine that induces an immune response. This response effectively maintains a low titer of virus or controls the viral rebound when the antiretrovial therapy is discontinued,, permitting significant reduction of the patient's dependency on antiretroviral therapy. While some such vaccines have been suggested as useful for seropositive patients (U.S. Pat. No. 5,863,542 column 18, lines 60-63), the art has not recognized that administration to seropositive patients receiving anti-viral treatment permits cessation of the anti-viral treatment without virus rebound, with delayed virus rebound, or with decreased post-rebound set point.

The present invention thus provides a method of control of virus rebound in HIV-infected patients after discontinuation of the antiviral therapy. By “control of virus rebound” we mean that after discontinuation of antiviral therapy the viral rebound that usually appears is delayed, the post-rebound set point is decreased, or there is no virus rebound.

Virus rebound appears usually within 1 to 3 weeks after discontinuation of the antiviral therapy. For the purposes of this invention, virus rebound is “delayed” when it appears more than 1 month after discontinuation of the antiviral therapy. Preferably the virus rebound appears more than 2 months and more preferably more than 6 months after discontinuation of the antiviral therapy.

The set point is defined as the plasmatic viral load that is maintained after viral rebound in the absence of antiviral treatment.

Viral rebound can be evaluated by various methods well known in the art. There are a variety of ways to measure viral titer in a patient. A review of the state of the art can be found in the “Report of the NIH to Define Principles of Therapy of HIV Infection” as published in the Morbidity and Mortality Weekly Reports, Apr. 24, 1998, Vol. 47, No. RR-5, Revised Jun. 17, 1998. It is known that HIV replication rates in infected persons can be accurately gauged by measurement of plasma HIV concentrations.

HIV RNA in plasma is contained within circulating virus particles or virions, with each virion containing two copies of HIV genomic RNA. Plasma HIV RNA concentrations can be quantified by target amplification methods (e.g., quantitative 13 RT polymerase chain reaction [RT-PCR], Amplicor HIV Monitor assay, Roche Molecular Systems; or nucleic acid sequence-based amplification, [NASBAS®], NucliSensam™ HIV-1 QT assay, Organon Teknika) or signal amplification methods (e.g., branched DNA [bDNA], Quantiplex™ HIV RNA bDNA assay, Chiron Diagnostics). The bDNA signal amplification method amplifies the signal obtained from a captured HIV RNA target by using sequential oligonucleotide hybridization steps, whereas the RT-PCR and NASBA® assays use enzymatic methods to amplify the target HIV RNA into measurable amounts of nucleic acid product. Target HIV RNA sequences are quantitated by comparison with internal or external reference standards, depending upon the assay used.

The method of vaccination of the invention is useful for the treatment of HIV-infected patients undergoing an antiretroviral therapy and having a viral load of less than 10,000, preferably less than 5,000, and more preferably less than 1000 viral copies per ml of plasma and a CD4+ T-cell count of above 300 cells/ml, preferably above 500 cells/ml.

By “antiretroviral therapy” or “antiviral therapy” we mean a treatment involving a potent combination of antiviral agents. Antiviral retroviral treatment involves the use of two broad categories of therapeutics. They are reverse transcriptase inhibitors and protease inhibitors. There are two type of reverse transcriptase inhibitors: nucleoside analog reverse transcriptase inhibitors and non-nucleoside reverse transcriptase inhibitors. Both types of inhibitors block infection by blocking the activity of the HIV reverse transcriptase, the viral enzyme that translates HIV RNA into DNA that can later be incorporated into the host cell chromosomes. Nucleoside and nucleotide analogs mimic natural nucleotides, molecules that act as the building blocks of DNA and RNA. Both nucleoside and nucleotide analogs must undergo phosphorylation by cellular enzymes to become active; however, nucleotide analogs used are already partially phosphorylated and is one step closer to activation when it enters a cell. Following phosphorylation, the compounds compete with the natural nucleotides for incorporation by HIV's reverse transcriptase enzyme into newly synthesized viral DNA chains, resulting in chain termination. Examples of anti-retroviral nucleoside analogs are: AZT, ddI ddC, d4T, and 3TC in combination with AZT and Combivir.

Non-nucleoside reverse transcriptase inhibitors (NNRTIs) are a structurally and chemically dissimilar group of anti-retrovirals. They are a highly selective inhibitors of HIV-1 reverse transcriptase. At present these compounds do not affect other retroviral reverse transcriptase enzymes such as those from hepatitis viruses, herpes viruses, HIV-2, and mammalian enzyme systems. They are used effectively in triple-therapy regimens. Examples of NNRTIs are Delavirdine and Nevirapine which have been approved for clinical use in combination with nucleoside analogs for treatment of HIV-infected adults who experience clinical or immunologic deterioration. A detailed review can be found in “Non-nucleoside Reverse Transcriptase Inhibitors” A-IDS Clinical Care (10197) Vol. 9, No. 10, p. 75.

Proteases inhibitors are compositions that inhibit HIV protease, which is a protease that is virally encoded and necessary for the infection process to proceed.

Clinicians in the United States have a number of clinically effective protease inhibitors to use on HIV infected persons. These include: SAQUINAVIR (Invirase); INDINAVIR (Crixivan); and RITONAVIR (Norvir).

Patients' viral load can be evaluated by various ways. Various methods which can be used have been disclosed above in relation with the virus rebound.

To assess a patient's immune system before antiviral treatment and after treatment as well as to determine if the claimed vaccine regimen is working, it is important to measure CD4+ T-cell counts. A detailed description of this procedure was published by Janet K. A. Nicholson, Ph.D et al., “1997 Revised Guidelines for Performing CD4+ T-Cell Determinations in Persons Infected with Human Immunodeficiency Virus (HIV)” in The Morbidity and Mortality Weekly Report, 46(RR-2): [inclusive page numbers], Feb. 14, 1997. Centers for Disease Control.

In brief, most laboratories measure absolute CD4+ T-cell levels in whole blood by a multi-platform, three-stage process. The CD4+ T-cell number is the product of three laboratory techniques: the white blood cell (WBC) count; the percentage of WBCs that are lymphocytes (differential); and the percentage of lymphocytes that are CD4+ T-cells. The last stage in the process of measuring the percentage of CD4+ T-lymphocytes in the whole-blood sample is referred to as “immunophenotyping by flow cytometry.” Immunophenotyping refers to the detection of antigenic determinants (which are unique to particular cell types) on the surface of WBCs using antigen-specific monoclonal antibodies that have been labeled with a fluorescent dye or fluorochrome (e.g., phycoerythrin [PE] or fluorescein isothiocyanate [FITC]). The fluorochrome-labeled cells are analyzed by using a flow cytometer, which categorizes individual cells according to size, granularity, fluorochrome, and intensity of fluorescence. Size and granularity, detected by light scattering, characterize the types of WBCs (ie., granulocytes, monocytes, and lymphocytes). Fluorochrome-labeled antibodies distinguish C7 populations and subpopulations of WBCs. Systems for measuring CD4+ T-cells are commercially available. For example Becton Dickenson's FACSCount System automatically measure absolutes CD4+, CD8+, and CD3+ T lymphocytes. It is a self-contained system, incorporating instrument, reagents, and controls.

Patients that can be treated by the method of the invention thus include those newly infected with HIV who have undergone intense anti-retroviral therapy within a few months after infection resulting in a controlled viremia (who can be defined as individuals showing an incomplete Western Blot), as well as chronically-infected individuals undergoing an antiretroviral therapy. By “newly infected” we mean patients who have been infected 90 or fewer days. By “controlled viremia” we mean that the viral load is maintained at a level of less than 10,000 viral copies per ml of plasma.

A preferred population of retrovirally infected persons are those that exhibit CD4+ and CD8+ cell response to HIV antigens, such as those that exhibit proliferative T-cell responses to envelope epitopes, e.g., HIV gp120.

More preferred are those patients that also respond to Gag epitopes, e.g., HIV p24. Typically these patients are identified by measuring the ability of their blood cells to proliferate in responses to highly purified antigen. In brief, peripheral blood monocytes (PBMC) are collected and cultured in the absence of IL-2 and in the presence of 10 μg of highly purified antigen. After four days the cultures are harvested and proliferation is measured by uptake of radioactive thymidine.

An alternative means is to use a skin test. Skin tests involve the detection of a delayed type hypersensitive response (DTH) by means of injecting or scratching antigen beneath the surface of the skin. The reaction is measured by the ability or inability of a patient to exhibit hypersensitive response to an aqueous solution of a gp120 or p24 antigen. Approximately, 1-20 μg is applied. The reaction is determined by measuring wheat sizes from about 24 to about 72 hours after administration of a sample, and more preferably from about 48 hours to about 72 hours after administration of a sample. Preferred wheal sizes for evaluation of the hypersensitivity of a patient range from about 16 mm to about 8 mm, more preferably from about 15 mm to about 9 mm., and even more preferably from about 14 mm to about 10 mm in diameter.

The method comprises administering to an HIV-infected patient as defined above a nucleic acid-based vaccine that enters the cells and intracellularly produces HIV-specific immunogens for presentation on the cell's MHC class I and MHC class II molecules in an amount sufficient to stimulate efficient HIV-specific CD4+ and CD8+ T-cell responses.

“Efficient CD8+ responses” is referred to as the ability of cytotoxic CD8+ T-cells to recognize and kill cells expressing foreign peptides in the context of a major histocompatibility complex (MHC) class I molecule. CD8+ T-cell responses may be measured, for example, by using tetramer staining of fresh or cultured PBMC, INF-γ Elispot assays, a combination of cell surface phenotyping and cytokine intracellular fluorescence staining intracellular INF-γor using functional cytotoxicity assays, which are well-known to those of skill in the art.

Briefly, for CTL assays, peripheral blood lymphocytes from patients are cultured with HIV peptide epitope at a density of about five million cells/ml. Following three days of culture, the medium is supplemented with human IL-2 at 20 units/ml and the cultures are maintained for four additional days. PBLs are centrifuiged over Ficoll-Hypaque and assessed as effector cells in a standard Cr-release assay using U-bottomed microtiter plates containing about 10⁴ target cells with varying effector cell concentrations. All cells are assayed twice. Autologous B lymphoblastoid cell lines are used as target cells and are loaded with peptide by incubation overnight during ⁵¹Cr labeling. Specific release is calculated in the following manner: (experimental release-spontaneous release)/(maximinum release-spontaneous release)×100. Spontaneous release is generally less than 20% of maximal release with detergent (2% Triton X-100) in all assays. “Efficient CD4+ responses” is referred to as the ability of CD4+ T-cells to be stimulated or activated by the vaccine of the invention. CD4+ T cell responses can be measured by various methods well-known in the art.

“Nucleic acid-based vaccine” means DNA and RNA-based vaccines and includes naked nucleic acids and vectored nucleic acids. By “vectored nucleic acid” we mean any kind of viral expression vectors such as DNA and RNA viruses or bacterial vectors such as BCG, salmonella or listeria or lactobacillus that delivers nucleic acid sequences coding for HIV specific immunogen into cells. The vectored nucleic acid corresponds preferably to an attenuated recombinant DNA virus.

“Attenuated recombinant virus” refers to a virus that has been genetically altered by modem molecular biological methods, e.g., restriction endonuclease and ligase treatment, and rendered less virulent than wild type, typically by deletion of specific genes or by serial passage in a non-natural host cell line permissive primary cells or at cold temperatures.

The selection of the virus to be used in the vaccine of the invention is not critical. Examples of viral expression vectors include adenoviruses as described in M. Eloit et al., “Construction of a Defective Adenovirus Vector Expressing the Pseudorabies Virus Glycoprotein gp50 and its Use as a Live Vaccine”, J. Gen. Virol., 71(10):2425-2431 (October, 1990).), adeno-associated viruses (see, e.g., Samulskl et al., J. Virol. 61:3096-3101 (1987); Samulski et al., J. Virol. 63:3822-3828 (1989)), papillomavirus, Epstein Barr virus (EBV) and Rhinoviruses (see, e.g., U.S. Pat. No., 5,714,374). Human influenza viruses are also reported to be useful, especially JS CP45 HPIV-3 strain. The viral vector may be derived from herpes simplex virus (HSV) in which, for example, the gene encoding glycoprotein H (gH) has been inactivated or deleted. Other suitable viral vectors include for example retroviruses (see, e.g., Miller, Human Gene Ther. 1:5-14 (1990); Ausubel et al., Current Protocols in Molecular Biology), coksackie viruses, vesicular stomatitis viruses (VSV) and poxviruses.

The poxviruses are preferred for use in this invention. There are a variety of attenuated poxviruses that are available for use as a vaccine against HIV. These include attenuated vaccinia virus, fowlpox virus and canarypox virus. These recombinant virus can be easily constructed. In brief, the basic technique of inserting foreign genes into live infectious poxvirus involves a recombination between poxvirus DNA sequences flanking a foreign genetic element in a donor plasmid and a homologous sequences present in the rescuing poxvirus as described in Piccini et aL, Methods in Enzymology 153, 545-563 (1987). More specifically, the recombinant poxviruses are constructed in two steps known in the art and analogous to the methods for creating synthetic recombinants of poxviruses such as the vaccinia virus and avipox virus described in U.S. Pat. Nos. 4,769,330, 4,722,848, 4,603,112, 5,110,587, and 5,174,993, the disclosures of which are incorporated herein by reference.

First, the DNA gene sequence encoding an antigenic sequence such as a known T-cell epitope is selected to be inserted into the virus and is placed into an E. coli plasmid construct into which DNA homologous to a section of DNA of the poxvirus has been inserted. Separately, the DNA gene sequence to be inserted is ligated to a promoter. The promoter-gene linkage is positioned in the plasmid construct so that the promoter-gene linkage is flanked on both ends by DNA homologous to a DNA sequence flanking a region of poxvirus DNA containing a nonessential locus. The resulting plasmid construct is then amplified by growth within E. coli bacteria Second, the isolated plasmid containing the DNA gene sequence to be inserted is transfected into a cell culture, e.g. chick embryo fibroblasts, along with the poxvirus. Recombination between homologous pox DNA in the plasmid and the viral genome gives a poxvirus modified by the presence of foreign DNA sequences in a non-essential region of its genome.

Attenuated recombinant pox viruses are employed in a preferred vaccine. A detailed review of this technology is found in U.S. Pat. No. 5,863,542 which is incorporated by reference herein. Representative examples of recombinant pox viruses include recombinant ALVAC and NYVAC. One example of recombinant ALVAC is vCP205. These viruses were deposited under the terms of the Budapest Treaty with the American Type Culture Collection (ATCC), 12301 Parklawn Drive, Rockville, Md., 20852, USA: NYVAC under ATCC accession number VR-2559 on Mar. 6, 1997; vCP205 (ALVAC-MiNI20TMG) under ATCC accession number VR-2557 on Mar. 6,1997; and, ALVAC under ATCC accession number VR-2547 on Nov. 14, 1996.

NYVAC is a genetically engineered vaccinia virus strain generated by the specific deletion of eighteen open reading frames encoding gene products associated with virulence and host range. NYVAC is highly attenuated by a number of criteria including: i) decreased virulence after intracerebral inoculation in newborn mice, ii) inocuity in genetically (nu⁺/nu⁺) or chemically (cyclophosphamide) immunocompromised mice, iii) failure to cause disseminated infection in immunocompromised mice, iv) lack of significant induration and ulceration on rabbit skin, v) rapid clearance from the site of inoculation, and vi) greatly reduced replication competency on a number of tissue culture cell lines including those of human origin.

ALVAC is an attenuated canarypox virus-based vector that was a plaque-cloned derivative of the licensed canarypox vaccine, Kanapox (Tartaglia et al., 1992). ALVAC has some general properties which are the same as some general properties of Kanapox.

ALVAC-based recombinant viruses expressing extrinsic immunogens have also been demonstrated efficacious as vaccine vectors. This avipox vector is restricted to avian species for productive replication. On human cell cultures, canarypox virus replication is aborted early in the viral replication cycle prior to viral DNA synthesis. Nevertheless, when engineered to express extrinsic immunogens, authentic expression and processing is observed in vitro in mammalian cells and inoculation into numerous mammalian species induces antibody and cellular immune responses to the extrinsic immunogen and confers protection against challenge with the cognate pathogen.

NYVAC and ALVAC have also been recognized as unique among all poxvirmses in that the National Institutes of Health (“NIH”)(U.S. Public Health Service), Recombinant DNA Advisory Committee (which issues guidelines for the safety containment of genetic material such as viruses and vectors, i.e., guidelines for safety procedures for the use of such viruses and vectors that are based upon the pathogenicity of the particular virus or vector) granted a reduction in physical containment level: from BSL2 to BSL1. No other poxvirus has a BSL1physical containment level. Even the Copenhagen strain of vaccinia virus (the common smallpox vaccine) has a higher physical containment level; namely, BSL2. Accordingly, the NIH has recognized that NYVAC and ALVAC have a lower pathogenicity than any other poxvirus.

Another attenuated poxvirms of preferred use in the invention is Modified Vaccinia virus Ankara (MVA), which acquired defects in its replication ability in humans as well as most mammalian cells following over 500 serial passages in chicken fibroblasts (see, e.g. Mayr et al, Infection 3:6-14 (1975); Carrol, M. and Moss, B. Virology 238:198-211 (1997)). MVA retains its original immunogenicity and its variola-protective effect and no longer has any virulence or contagiousness for animals and humans. As in the case of NYVAC and ALVAC, expression of recombinant protein occurs during an abortive infection of human cells, thus providing a safe, yet effective, delivery system for foreign antigens.

The nucleic acid-based vaccine for use in the present invention further comprises sequences encoding HIV immunogens and intracellularly produces the HIV-specific immunogens. The HIV antigen encoding DNA for insertion into the viral vectors of the invention or for use as naked nucleic acid are any that are known to be effective for protection against a retrovirus. “HIV-specific immunogens” means any HIV protein, fragment, or epitope thereof that is recognized by an immune cell as an epitope of the native protein. HIV-specific immunogens are thus selected from both structural and non-structural proteins. Highly antigenic epitopes for provoking an immune response selective for a specific retroviral pathogen are known.

“Nonstructural viral proteins” are those proteins that are needed for viral production but are not necessarily found as components of the viral particle. They include DNA binding proteins and enzymes that are encoded by viral genes but which are not present in the virions. Proteins are meant to include both the intact proteins and fragments of the proteins or peptides which are recognized by the immune cell as epitopes of the native protein.

“Structural viral proteins” are those proteins that are physically present in the virus. They include the envelope, the capsid proteins, and enzymes that are loaded into the capsid with the genetic material. Because these proteins are exposed to the immune system in high concentrations, they are considered to be the proteins most likely to provide an antigenic and immunogenic response. Proteins are meant to include both the intact proteins and fragments of the proteins or peptides which are recognized by the immune cell as epitopes of the native protein.

The envelope is a preferred source of epitopes and gp 160, 120 and 41 are sources of immunoprotective proteins. Both B and T cell epitopes have been described in the literature and can be used. Peptides selected from the V3 loop of the HIV envelope proteins are of preferred use. In addition other structural proteins have been reported to be imnmunoprotective including gp41 and the Gag protein. By “Gag protein” we mean the whole Gag protein as well as proteins derived from Gag such as p17 and p24. Non-structural genes include the rev, tat, nef vif, and vpr genes.

For HIV, the nucleic acids include those that can code for at least one of -HIV-I Gag(+pro)(LAI), gp120(MN or another strain)(+transmembrane), Nef(BRU)CTL, Pol(IIIB)CTL, ELDKWA or LDKW epitopes, preferably HIV 1 Gag(+pro)(IIIB), gp120(MN) (+transmembrane), two (2) Nef(BRU)CTL and three (3) Pol(III)CTL epitopes; or two ELDKWA in gp120 V3 or another region of gp160. The two (2) Nef(BRU)CTL and three (3) Pol(IIIB)CTL epitopes are preferably Nef1, Nef2, Pol1, Pol2 and Pol3. The corresponding sequences are given in U.S. Pat. No. 5,990,091. Furthermore, sequences encoding Tat and/or Rev can advantageously be added. In the above listing, the viral strains from which the antigens are derived are noted parenthetically. The above-defined HIV antigen encoding DNA can be derived from any known HIV strain (HIV1, HIV2, preferably HIV 1), including laboratory strains and primary isolates.

The Pol and Nef epitopes have sequences presented in the following:

| | |
|---|---|
| MPLTEEAELE LAENREILKE PVHGVYYDPS KDLIAEIQKQ GQGQWTYQIY QEPFKNLKTG | 60 |
| MEWRFDSRLA FHHVARELHP EYFKNCKLMA IFQSSMTKIL EPFRKQNPDI VIYQYMDDLY | 120 |
| VGSDLEIGQH RTKIEELRQH LLRWGLTTMV GFPVTPQVPL RPMTYKAAVD LSHFLKEKGG | 180 |
| LEGLIHSQRR QDILDLWIYH TQGYFPDWQN YTPGPGVRYP LTFGWCYKLV PMIETVPVKL | 240 |
| KPGMDGPKVK QWPLTEEKIK ALVEICTEME KEGKISKIGP 280 | |

where

1-60: CTL epitope Pol-3 (60 aa)

61-86: CTL epitope Nef-2 (26 aa)

89-148: CTL epitope Pol-2 (60 aa)

149-231: CTL epitope Nef-1 (83 aa)

232-280: CTL epitope Pol-1 (49 aa)

Preferred viral vectors according to the invention include ALVAC HIV (vCP1452), which is a recombinant canarypox virus expressing Gag_{LAI}, Protease_{LAI}, Env(120)_{MN}, Env(41)_{LAI}, Nef, and Pol. vCP1452 is described in U.S. Pat. Nos. 6,004,777 and 5,990,091. Also useful in the invention is vCP1433, which was deposited with the ATCC in accordance with the Budapest Treaty on March 6, 1997, under accession number VR-2556 and was also described in U.S. Pat. Nos. 6,004,777 and 5,990,091.

Other vectors useful in the invention include those in the table below:

| ALVAC-HIV | Inserted HIV genes |
|---|---|
| vCP125* | gp160_{MN} |
| vCP205** | gp120_{MN} and portion gp41_{LAI}, gag_{LAI}, and protease_{LAI} |
| vCP300*** | gp120_{MN} and portion gp41_{LAI}, gag_{LAI}, and protease_{LAI} |
| | and pol CTL domains: 172-219, 325-383, 461-519, nef |
| | CTL domains: 66-147, 182-206 |

*As described in U.S. Pat. No. 5,766,598.
**ALVAC-MN120TMG deposited on Mar. 6, 1997 as ATCC accession number VR-2557)
***As described in U.S. Pat. No. 5,863,542.

The administration procedure for recombinant virus and DNA is not critical. Vaccine compositions (e.g., compositions containing the poxvirus recombinants or DNA) can be formulated in accordance with standard techniques well known to those skilled in the pharmaceutical art. Vaccine compositions can comprise one or a plurality of vectors that effect HIV-antigen expression. Such compositions can be administered in dosages and by techniques well known to those skilled in the medical arts taking into consideration such factors as the age, sex, weight, and condition of the particular patient, and the route of administration.

Vaccines may be delivered via a variety of routes of administration including, for example, a parenteral route (intradermal, intramuscular or subcutaneous, transdermal or epidermal). Other routes include oral administration, intranasal, intrarectal and intravaginal routes. Examples of vaccine compositions of use for the invention include liquid preparations, for orifice, e.g., oral, nasal, anal, vaginal, etc. administration, such as suspensions, syrups or elixirs; and, preparations for parenteral, subcutaneous, intradermal, intramuscular or intravenous administration (e.g., injectable administration) such as sterile suspensions or emulsions. In such vaccines the naked or vectored nucleic acid may be in admixture with a suitable carrier, diluent, or excipient such as sterile water, physiological saline, glucose, Tris buffer or the like. The vaccine of the invention may also comprise an adjuvant. Any adjuvant administrable to humans can be used. Adjuvants useful in the invention include alum, calcium phosphate and, preferably PCPP (poly dicarboxylatopheoxylphosphazene), a synthetic hydrogel polymer developed for its adjuvant properties.

A viral vector-based vaccine can be administered at about 10³-10⁸ TCID50/dose or 10⁴to 10⁹ pfu per dose. For example, ALVAC-HIV vaccine is inoculated, more than once, by the intramuscular route at a dose of about 10⁸ pfu per inoculation, for a patient of 170 pounds. The vaccine may be delivered in a physiologically compatible solution such as sterile 0.4% NaCl in a volume of, e.g., one ml. The vaccine of the invention is administered several times. Intervals between administrations and number of administration depend of the immune response of the patient. Vaccine doses have to be administered as long as it is necessary to re-induce the immune system. Actual dosages of such a vaccine can be readily determined by one of ordinary skill in the field of vaccine technology.

As an alternative to a viral vaccine, DNA may also be directly introduced into the cells of a patient. This embodiment is defined in the present invention as naked-DNA vaccine. This expression (i.e., naked-DNA vaccine) thus encompasses naked DNA per se, including virus like particles, as well as formulated DNA-based vaccines as disclosed below. This approach is described, for instance, in Wolff et. al, Science 247:1465 (1990) as well as U.S. Pat. Nos. 5,580,859; 5,589,466; 5,804,566; 5,739,118; 5,736,524; 5,679,647; and WO 98/04720. Examples of DNA-based delivery technologies include, “naked DNA,” facilitated (bupivicaine, polymers, peptide-mediated, adjuvants) delivery, and cationic lipid complexes or liposomes and microspheres. Alternatively, the nucleic acids can be administered using ballistic delivery as described, for instance, in U.S. Pat. No. 5,204,253 or pressure (see, e.g. U.S. Pat. No. 5,922,687). Using this technique, particles comprised solely of DNA are admninistered. In a further alternative embodiment, DNA can be adhered to particles, such as gold particles. As is well known in the art, a large number of factors can influence the efficiency of expression of antigen genes and/or the inununogenicity of DNA vaccines. Examples of such factors include the reproducibility of inoculation, construction of the plasmid vector, choice of the promoter used to drive antigen gene expression and stability of the inserted gene in the plasmid. Any of the conventional vectors used for expression in eukaryotic cells may be used for directly introducing DNA into tissue. Expression vectors containing regulatory elements from eukaryotic viruses are typically used in eukaryotic expression vectors, e.g. CMV vectors. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 later promoter, metallothionein promoter, human cytomegalovirus promoter, murine mammary tumor virus promoter, Rous sarcoma virus promoter, polyhedrin promoter, or other promoters shown effective for expression in eukaryotic cells.

Therapeutic quantities of plasmid DNA can be produced, for example, by fermentation in E. coli followed by purification. Aliquots from the working cell bank are used to inoculate growth medium and grown to saturation in shaker flasks or a bioreactor according to well known techniques. Plasmid DNA can be purified using standard bioseparation technologies such as solid phase anion-exchange resins supplied by QIAGEN, Inc. (Valencia, Calif.). If required, supercoiled DNA can be isolated from the open circular and linear forms using gel electrophoresis or other methods.

Purified plasmid DNA can be prepared for injection using a variety of formulations. The simplest of these is reconstitution of lyophilized DNA in sterile phosphate-buffer saline (PBS). This approach, known as “naked DNA,” is currently being used for intramuscular (IM) administration in clinical trials.

To maximize the immunotherapeutic effects of minigene DNA vaccines, an altemative method for formulating purified plasmid DNA may be desirable. A variety of methods have been described, and new techniques may become available. Cationic lipids can also be used in the formulation (e.g., as described by WO 93/24640; Mannino & Gould-Fogen'te, BioTechniques 6(7): 682 (1988); U.S. Pat No. 5,279,833; WO 91/06309; and Felper, et al., Proc. Nat'l Acad. Sci. USA 84:7413 (1987). In addition, glycolipids, fusogenic liposomes, peptides targeting sequences and compounds referred to collectively as protective, interactive, non-condensing compounds could also be complexed to purified plasmid DNA to influence variables such as stability, intramuscular dispersion, or trafficking to specific organs or cell types. DNA expression vectors for direct introduction of DNA into the patient tissue can additionally be complexed with other components such as peptides, polypeptides, lipopeptides, carbohydrates, microspheres, immunostimulants and adjuvants. Expression vectors can also be complexed to particles or beads that can be administered to an individual, for example, using a vaccine gun.

The expression vectors are administered by methods well known in the art as described, for example, in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al (U.S. Pat. No. 5,679,647, issued Oct. 21, 1997), each of which is incorporated herein by reference. One skilled in the art would know that the choice of a pharmaceutically acceptable carrier, including a physiologically acceptable compound, depends, for example, on the route of administration of the expression vector.

For example, naked DNA or polynucleofide in an aqueous carrier can be injected into tissue, such as muscle or skin, in amounts of from 101 per site to about 1 ml per site. The concentration of polynucleotide in the formulation is from about 0.1 μg/ml to about 20 mg/ml. Actual dosages of the vaccine can be readily determined by one of ordinary skill in the field of vaccine technology.

The expression vectors of use for the invention can be delivered to the interstitial spaces of tissues of an animal body (Felgner et al., U.S. Pat. Nos. 5,580,859 and 5,703,055). Administration of expression vectors of the invention to muscle is a particularly effective method of administration, including intradermal and subcutaneous injections and transdermal administration. Transderrnal administration, such as by ionophoresis, is also an effective method to deliver expression vectors of the invention to muscle. Epidermal administration of expression vectors of the invention can also be employed. Epidermal administration involves mechanically or chemically irritating the outermost layer of epidermis to stimulate an immune response to the irritant (Carson et al., U.S. Pat. No. 5,679,647). The vaccines can also be formulated for administration via the nasal passages. Formulations suitable for nasal administration, wherein the carrier is a solid, include a coarse powder having a particle size, for example, in the range of about 10 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active ingredient. For further discussions of nasal administration of AIDS-related vaccines, references are made to the following patents, U.S. Pat. No. 5,846,978, 5,663,169, 5,578,597, 5,502,060, 5,476,874, 5,413,999, 5,308,854, 5,192,668, and 5,187,074.

The vaccines can be incorporated, if desired, into liposomes, microspheres or other polymer matrices (Feigner et al., U.S. Pat. No. 5,703,055; Gregoriadis, Liposome Technology, Vols. I to III (2nd ed. 1993), each of which is incorporated herein by reference). Liposomes, for example, which consist of phospholipids or other lipids, are nontoxic, physiologically acceptable and metabolizable carriers that are relatively simple to make and administer.

Liposome carriers may serve to target a particular tissue or infected cells, as well as increase the half-life of the vaccine. Liposomes include emulsions, foams, micelles, insoluble monolayers, liquid crystals, phospholipid dispersions, lamellar layers and the like. In these preparations the vaccine to be delivered is incorporated as part of a liposome, alone or in conjunction with a targeting molecule which binds to, e.g., a receptor prevalent among lymphoid cells, such as monoclonal antibodies which bind to the CD45 antigen, or with other therapeutic or immunogenic compositions. Thus, liposomes either filled or decorated with a desired immunogen of the invention can be directed to the site of lymphoid cells, where the liposomes then deliver the immunogen(s).

Liposomes for use in the invention are formed from standard vesicle-forming lipids, which generally include neutral and negatively charged phospholipids and a sterol such as cholesterol. The selection of lipids is generally guided by consideration of, e.g., liposome size, acid lability and stability of the liposomes in the blood stream. A variety of methods are available for preparing liposomes, as described. in, e.g., Szoka, et al., Ann. Rev. Biophys. Bioeng. 9:467 (1980), U.S. Pat. Nos. 4,235,871, 4,501,728, 4,837,028, and 5,019,369.

Vaccines for use in the present invention can be administered alone or can advantageously be combined with an immunostimulating composition and/or another anti-HIV vaccine.

By “combined” we mean a simultaneous or a sequential administration (e.g. prime-boost) of a vaccine and an immunostimulating composition and/or of another anti-HIV vaccine.

Vaccines for use in the invention can advantageously be combined with immunostimulatory or co-stimulatory molecules such as for example cytokines, interleukin 2 or CD40 ligand, which are-used in an amount that is sufficient to potentiate the T-cell responses, in particular CD8+ responses. These immunostimulating compounds are used according to the recommendations of the manufacturer. Such compounds may be present as such or in the form of a recombinant virus expressing the same.

Vaccines for use in the invention can advantageously be combined with another anti-HIV vaccine. Such anti-HIV vaccine can be different from the first vaccine (for example, naked nucleic acid-based vaccine can be combined with a viral vector-based vaccine, naked DNA followed by a HIV immunogen-encoding poxvirus, or an HIV-immunogen encoding attenuated vaccinia virus followed by a HIV immunogen-encoding avipox virus), or can be a vaccine comprising a soluble antigen of HIV. Any soluble HIV antigen that is known to be an effective antigen for protection against HIV can be used. According to a preferred embodiment, the soluble antigen corresponds to the gp160 HIV-1 envelope glycoprotein and, in particular, the gp160MN/LAI-2, corresponding to an envelope glycoprotein from HIV-1 virus expressed by vaccinia virus VV.TG.9150 on BHK₁ cells wherein the gp120 portion is derived from HIV_{MN} and the gp41 transmembrane portion from HIV_{LAI}. Actual dosages of the soluble antigen can be readily determined by one of ordinary skill in the field of vaccine technology

According to a preferred embodiment, the vaccine comprises a nucleic acid vector (e.g., a viral vector) comprising genes encoding and expressing a plurality of HIV antigens and is co-administered with an HIV antigen. In a most preferred embodiment, a vector comprising the ALVAC canarypox vector expressing the HIV Gag, Protease, Env(120), Env (41), Nef, and Pol antigens is co-administered with the gp160 HIV-1 envelope glycoprotein.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### EXAMPLES

Haart Therapy

Ongoing HAART clinical trials at the Aaron Diamond AIDS Research Center are summarized in Table 1:

TABLE 1
Clinical Trials at the ADARC of the Rockefeller University

| | | | | |
|---|---|---|---|---|
| | | | | Duration of |
| Study | Treatment | # active/# | Study | therapy |
| Identifier | Regimen | recruited | Population | (mos.) |

| | | | | |
|---|---|---|---|---|
| MMA-160 | AZT/3TC/RIT | 8/12 | Newly infected | 26-32 |
| MMA-167 | AZT/3TC/IND | 11/12 | Newly infected | 19-25 |
| MMA-174 | AZT/3TC/NLF | 8/12 | Infection > 90 d | 27 |
| MMA-183 | RIT/SAQ | 10/12 | Infection > 90 d | 21 |
| MMA-197 | AZT/3TC/ | 12/14 | Newly infected | 13-20 |
| | RIT/SAQ | 10/13 | Inf. > 90 d | 19 |
| MMA-227 | AZT/3TC/ | 12/13 | Newly infected | 1-12 |
| | 1592/GW141 | 11/12 | Inf. > 90 d | 7-12 |

RIT = ritonavir
IND = indinavir
NLF = nelfinavir
SAQ = saquinavir
1592 = Abacavir
GW141 = Vertex 478 (Protease inhibitor)

The clinical program divided study subjects into two groups, those newly infected and those infected for greater than 90 days on entry into the screening phase.

New infections were diagnosed on the basis of a positive plasma HIV-1 RNA in the setting of one of the following three criteria: absence of HIV-antibody by ELISA, progression of the antibody response as determined by the appearance of at least two new bands on Western blot and a clinical syndrome consistent with acute infection within 90 days of screening, and a documented negative test within the previous 120 days.

Participants in these clinical trials were generally followed weekly for four weeks, bi-weekly for two months, then monthly to assess for both safety and efficacy. Routine laboratory determinations include plasma HIV-RNA levels using either bDNA signal amplification or PCR technology, safety laboratory studies including routine hematology and chemistry, and assessments of immunologic status including a variety of cell surface markers used to define naïve and memory cell subsets.

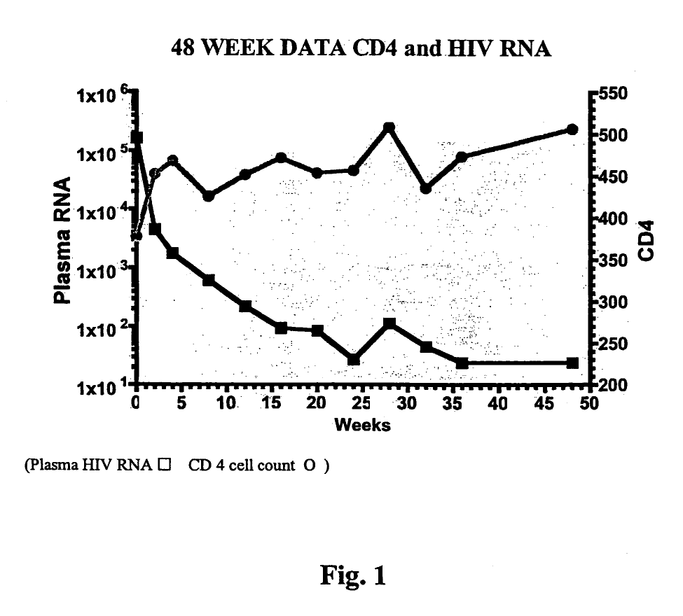

Representative longitudinal plasma HIV-RNA and CD4 cell data of a chronically infected cohort participating in study MMA-197 is shown in FIG. 1. As depicted in FIG. 1, suppression of virus replication is accompanied by a 2 log drop in HIV RNA during the early weeks. Further suppression of the productive infection of new susceptible cells results in a continued drop in the plasma HIV-1 RNA reflecting the loss of cells continuing to produce non-infectious virus particles. The antiviral effect is dramatic and results in a nearly 4 log reduction as the nadir is reached at week 24.

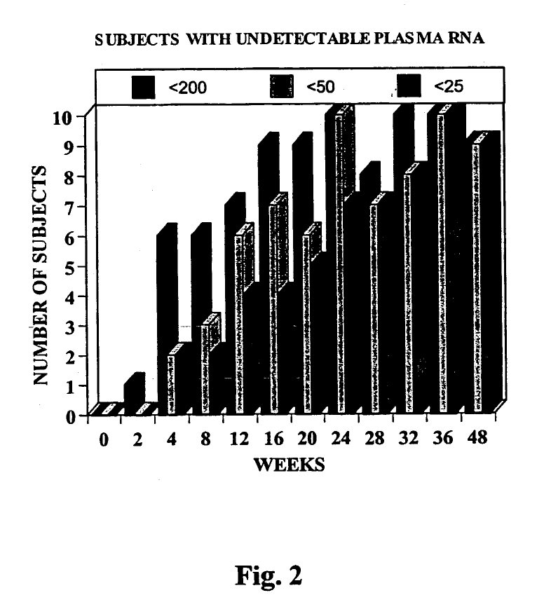

The impact of complete suppression of virus replication can be viewed in a somewhat different way in FIG. 2. As the weeks of therapy progress the level of HIV-RNA measured in this group of treated subjects becomes increasingly difficult to detect. By the end of 48 weeks, all of the subjects treated with this four drug combination met the goal of “undetectability.” These results suggest that the total pool of infected cells still producing particles at this time point has fallen to a very low level.

Lymphoid tissue was obtained from patients participating in these studies after a minimum of 12 months of HAART therapy. Gastrointestinal-associated-lymphoid tissue (GALT) was obtained in the majority of subjects. Biopsies were graded on a scale of 1 to 4; 1=scattered lymphoid cells, 2=small lymphoid aggregate, 3=large well defined aggregate, 4=germinal center present. Individuals also agreed to undergo tonsillar biopsy or lymph node biopsy. Eight subjects underwent gastrointestinal biopsy. In 4 in whom follicles were present, no trapped virus was detected. In all 8, a limited number of tissue sections examined did not reveal RNA expressing cells. With extensive sampling of the biopsied material from subject 9 the rare expression of HIV-specific RNA could be detected in rectal tissue, tonsil, and cervical node. Germinal centers were free of trapped virus and the rare RNA-positive cell had relatively few grains (7 to 37) compared to untreated controls in which the grain number was too numerous to count.

To maximize the detection of potentially infectious virus, we performed co-cultures of mononuclear cells (MC) from blood after depletion of CD8+ T-cells to remove potential inhibitory soluble factors and stimulated the MC with PHA.

Using the method of Saksela and Vesanen, nested PCR for both multiply-spliced (MS) and unspliced (U.S.) -HIV-mRNA and proviral DNA were performed on MC from blood and lymphoid tissue. The results of these studies in the peripheral blood from subjects participating in study MMA-160 is summarized in Table 2.

TABLE 2
Blood

| | | PHA | | | |
|---|---|---|---|---|---|
| | Duration | Stimulated | RNA PCR | | |
| | of | Culture | multiply | RNA PCR | DNA PCR |
| | Therapy | TCID₅₀/10⁶ | spliced | Unspliced | (copies/10⁶ |
| Subject | (months) | CD8-CD4) | copies/mg | copies/mg | PBMC) |
| 2 | 24 | <0.1 | <50 | 372 | 353 |
| 3 | 23 | <0.1 | <50 | 459 | 503 |
| 5 | 22 | >0.1 | <50 | 1921 | 528 |
| 6 | 20 | >0.1 | <50 | 295 | 254 |
| 7 | 20 | <0.1 | <50 | 167 | 167 |
| 8 | 19 | <0.1 | <50 | 284 | 284 |
| 9 | 19 | >0.1 | <50 | 112 | 112 |
| 11 | 18 | >0.1 | 312 | 1521 | 1753 |

Viral load of CD8+ T cell-depleted PHA-stimulated co-cultures after 19 to 24 months of therapy were less than 0.1 TCID₅₀/10⁶ CD4 in Subjects 2, 3, 7, and 8. Cultures were strongly positive in subjects 6, 9 and 11 and borderline positive in Subject 5. Quantitative PCR detected both MS and US-MRNA in PBMC from subject 11.

US-mRNA was detected in PBMC from subjects 2,3, 5 and 6. PBMC from subjects 7, 8, and 9 did not reveal detectable mRNA.

Culture and quantitative PCR results for GALT and other lymphoid tissues obtained early in the second year of therapy in the same cohort of early treated subjects are shown in Table 3. These are compared to a control with high levels of virus replication in blood and lymphatic tissue.

TABLE 3
GALT

| | | | | | RNA PCR | |
|---|---|---|---|---|---|---|
| | | | Culture | RNA PCR | unspliced | DNA PCR |
| | Duration of Therapy | | (TCID₅₀/10⁶ | multiply spliced | copies/mg | (copies/10⁶ |
| Subject | (months) | Site of biopsy | PBMC) | copies/mg mRNA | mRNA | PBMC) |
| Positive control | N/A | desc. colon | 1 | 439 | 256,062 | 5,346 |
| | | sigmoid | | 375 | 194,338 | 2,230 |
| | | rectum | | 351 | 170,851 | 3,624 |
| 2 | 16 | desc. colon | <0.25 | <50 | <50 | 150 |
| | | sigmoid | | <50 | <50 | ND |
| | | rectum | | <50 | <50 | 121 |
| 3 | 17 | desc. colon | <0.1 | <50 | 331 | 10 |
| | | sigmoid | | <50 | <50 | 99 |
| | | rectum | | <50 | <50 | 117 |
| 5 | 16 | desc. colon | <1.0 | <50 | 155 | <10 |
| | | sigmoid | | <50 | 101 | ND |
| | | rectum | | <50 | 217 | 58 |
| 6 | 13 | desc. colon | <1.0 | <50 | 102 | ND |
| | | sigmoid | | <50 | <50 | 83 |
| | | rectum | | <50 | <50 | <10 |
| 7 | 15 | desc. colon | <0.1 | <50 | 110 | <10 |
| | | sigmoid | | <50 | <50 | 23 |
| | | rectum | | <50 | <50 | <10 |
| 8 | 14 | desc. colon | <0.1 | <50 | 101 | 260 |
| | | sigmoid | | <50 | <50 | 68 |
| | | rectum | | <50 | <50 | 540 |
| 9 | 12 | desc. colon | <0.1 | <50 | <50 | <10 |
| | | sigmoid | | <50 | 411 | <10 |
| | | rectum | | <50 | <50 | <10 |
| 9 | 15 | tonsil 1 | <0.1 | <50 | 345 | 76 |
| | | tonsil 2 | | <50 | 245 | 56 |
| | | lymph node 1 | <0.1 | <50 | 987 | 28 |
| | | lymph node 2 | | <50 | <50 | <10 |
| | | sigmoid 1 | <0.1 | <50 | <50 | <10 |
| | | sigmoid 2 | | <50 | 454 | 14 |
| | | rectum | | <50 | <50 | 38 |
| 11 | 13 | desc. colon | <0.1 | <50 | <50 | 150 |
| | | sigmoid | | <50 | <50 | <10 |
| | | rectum | | <50 | <50 | 207 |

Studies performed on GALT during months 12 to 17 are similarly presented. MC co-cultures were routinely below the level of detection as was the level of MS-mRNA. US-mRNA was detected in very low levels in all subjects except 2 and 11. Proviral DNA was routinely detected in the MC of all subjects. At the month 15 visit subject 9 underwent tonsil and cervical lymph node biopsy. Similar results are observed in these samples; no culturable virus, undetectable MS-mRNA, low level US-RNA expression, and low copy number of proviral DNA.

A lumbar puncture was performed in subjects 3 and 9 at months 24 and 15, respectively. In both, the fluid was acellular and had less than 25 HIV-RNA copies/ml as determined by ultra-sensitive RNA PCR (Roche).

Analysis of semen concurrent with lymnphoid tissue biopsy revealed mononuclear cells (MC) with undetectable levels of both multiply-spliced and unspliced HBV-mRNA. Proviral DNA was detected at low levels, between 10 and 100 copies/10⁶ MC in all but one subject (#9).

During various clinical studies intensive virologic measurement were performed in early infected HAART treated subjects. New infections were diagnosed on the basis of a positive plasma HIV-1 RNA in the setting of one of the following three criteria: absence of HIV-antibody by ELISA, progression of the antibody response as determined by the appearance of at least two new bands on Western blot and a clinical syndrome consistent with acute infection within 90 days of screening, and a documented negative test within the previous 120 days.

The results of these studies suggest that as these newly infected subjects reach the second year of therapy, there exists a minimal level of HIV-1 expression. It cannot be determined that HIV expression necessarily translates into ongoing rounds of infection of susceptible cells, but may represent stochastic activation of the latently infected population that is controlled by the presence of the antiviral regimen.

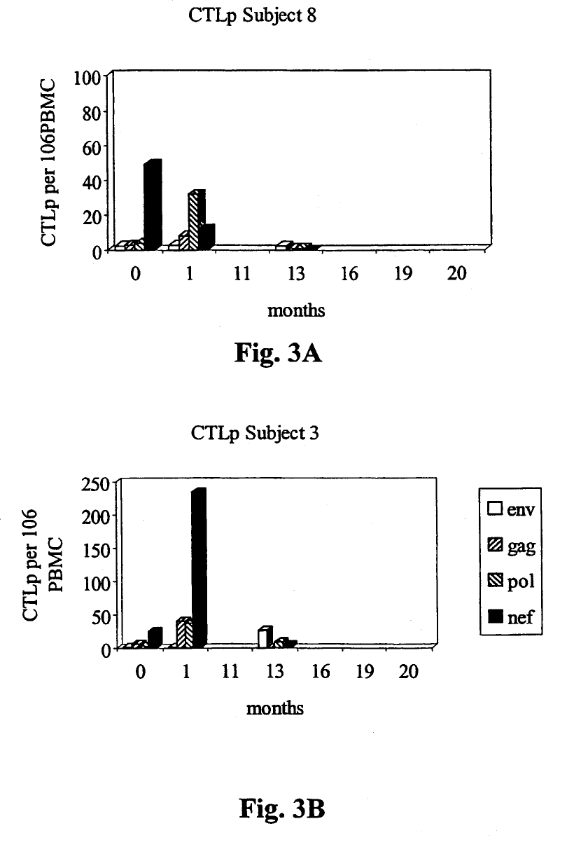

The reduction in total body virus burden has a significant effect on both CTL precursor frequencies and antibody levels to Gag and Env in this cohort of newly infected subjects. As seen in FIG. 3, subjects 3 and 8, levels of CTLp drop with time as HIV replication is inhibited. Similar results are seen in similarly treated subjects in both newly infected and chronically infected cohorts.

Similarly, persistent control of virus replication results in significant reductions in HIV-specific antibodies to Env (gp120) and Gag (p24). This has been observed, however, only in the newly infected and not the chronically infected treatment group.

Based on the low level of HIV-specific immune responses as a consequence of effective antiviral therapy and the small pool of latently infected cells harboring potentially infectious virus, we concluded that stimulation of HIV specific immune response would be desirable prior to discontinuation of antiviral therapy. We believed that based on the results from studies of newly infected subjects and long-term non-progressors with minimal virus activity, CD4+ T-cell and CTL activities are critical immunologic control factors. Other data suggested to us that high levels of neutralizing antibodies are associated with lack of disease progression.

Our vaccine strategy is based on the concept that both humoral and cell-mediated immune responses can be stimulated by stimulating the immune system with live recombinant vectors expressing various HIV-1 antigens and with soluble recombinant proteins as discussed above.

HIV Vaccine Research Design and Methods

Subjects already participating in ongoing HAART clinical trials conducted by the clinical arm of the Aaron Diamond AIDS Research Center were eligible for participation in this study.

A. Pre-entry Virologic Evaluation

HIV-infected subjects participating in one of the HAART clinical trials at ADARC (newly infected) underwent extensive virologic evaluation after a mininum of two years of therapy.

Blood, lymphoid tissue including tonsil and/or lymph node(Study#MMA-189), semen (#MMA-205), and CSF (#MMA-203) were collected on all consenting subjects. Participation required informed consent by signature for each procedure listed above. No subject was excluded from participation in this vaccine study based on participation in these other studies of tissue and body fluids (see inclusion/exclusion criteria, below).

Blood was processed as follows; plasma was separated by centrifugation and stored at −70° C. for subsequent studies as well as ultra-sensitive HIV-RNA determination using a modification of the Roche Amplicor assay.⁴³ This assay was the most sensitive and reproducible assay available to determine levels of HIV-1 RNA in plasma. Peripheral blood mononuclear cells (PBMC) are isolated by Ficoll-Hypaque gradient using standard techniques. Aliquots of a minimum of 10⁷ cells were prepared and stored at −150° C. for future use. Cells were CD8 depleted using magnetized-antibody-coated polystyrene beads (Dynal). 1-2×10⁷ CD8-depleted MC were stimulated with PHA and irradiated feeder cells and co-cultured in IL-2 containing medium with HIV-negative donor CD4+ T-cells. Cultures were maintained for three weeks and culture supernatants assayed weekly for levels of p24. A positive culture requires a p24 concentration of at least 100 pg/ml in the culture supernatant.

As the lymphoid system is the preferred site of virus replication in an infected host, a comprehensive surgical program was established at Rockefeller University Hospital to meet the specific needs of the ADARC clinical program. A general surgeon to perform inguinal lymph node biopsy and an otolaryngologist (ENT) to perform either cervical node or tonsillar biopsy were recruited. A board-eligible gastroenterologist obtained gastrointestinal-associated lyimphoid-tisgsue (GALT). These procedures were done under separate protocols MMA-189 and ATA-207.

Consenting subjects were well-known to the clinical staff, but screening for coagulopathy with measurements of prothrombin time (PT) and partial thromboplastin time (PTT) was included prior to procedure. A careful surgical history was also required to screen for rarer causes of hemostatic dysfunction. Biopsies were performed using local anesthesia without the need for conscious sedation. Lymphoid tissue was divided into three sections, a portion immediately frozen in liquid nitrogen for PCR analysis, a portion formalin-fixed and subsequently paraffin embedded for in situ hybridization and immunohistochemistry, and a portion transported in culture medium from which MC were mechanically disrupted and cultured using standard co-culture techniques.

All culture supernatants positive for HIV-RNA were analyzed for the presence of either genotypic or phenotypic resistant virus. Similarly, all plasma samples with HIV-1 RNA above 500 copies/ml were used for RT-PCR, although the limitations of this assay at low copy number was well appreciated.

Semi-quantitative PCR for multiply-spliced (MS) and unspliced (US)-mRNA as well as proviral DNA were performed on PBMC and MC from semen, cervical lavage, and lymphoid tissue with a modified technique of Vesanen and Saksela.⁴⁴⁻⁴⁶

Finally, paraffin embedded sections of lymphoid tissue were subject to in-situ hybridization pursuant to published techniques.^{47,48}

Subjects eligible for vaccination had to meet the following virologic criteria:

1. Undetectable levels of MS-mRNA in blood and/or tissue

2. Rare to no HIV expressing cells by in-situ hybridization (tissue sampling is optional).

3. Viral cultures from blood and/or tissue either negative for culturable virus or yielding drug-sensitive virus by genotype and phenotype.

Subjects failing to meet these virologic criteria could be re-evaluated at 6 month intervals.

B. HIV-Specific Immunologic Evaluation

Simultaneous immunologic investigations were performed after two years of therapy to determine eligibility for vaccination.

Direct CTL effector activity was measured from freshly isolated PBMC using autologous B-lymphoblastoid cell targets infected with recombinant vaccinia virus expressing HIV-1 specific genes (gag, pol, env, nef).⁴

HIV-specific CTL precursor frequencies (CTLp) were similarly performed in selected subjects.⁴⁹ Patient PBMC were seeded at varying concentrations in 200 μl of IL-2-containing medium in 24 replicate-wells of a 96-well tissue culture plate. Irradiated donor PBMC and anti-CD-3 antibody were added to each well and incubated at 37° C. for 14 days. Wells were split into four and assayed for the ability to lyse an autologous chromium-labeled B-lymphoblastoid cell line infected with a vaccinia-virus expressing HIV-1 env, gag, pol, and nef genes as well as an antigen negative control. CTLp with a given specificity were determined by plotting the log of the fraction of negative wells (less than 3 S.D. above the mean for the 24 control wells or below 10% specific lysis) versus the number of input cells.⁴

Patients with detectable fresh CTL activity above 30% specific lysis to one or more antigens at an effector to target ratio of 25:1 were not eligible for participation in the vaccination protocol. Subjects with levels of CTL precursors above 1 in 100,000 to one or more specific antigens including Env, Gag, Pol, or Nef were similarly excluded.

C. Inclusion Criteria

The following criteria were used to select patients for the vaccination study:

HIV infected subjects with at least 2 years of combination antiretroviral therapy

Plasma HIV-RNA<25 copies/ml

Absent Multiply Spliced (MS) RNA determinations in peripheral blood

Qualitative CD4 cell co-culture either negative or positive for wild-type virus (as determined by genotype) from blood

Ability to give informed consent

Age greater than 18

There were no CD4+ T cell count entry criteria ps D. Optional Entry Criteria

In subjects agreeing to tissue biopsy or body fluid collection (genital secretions, CSF), after 24 months of therapy, the following virologic criteria had to be met:

Absent Multiply Spliced (MS) RNA determinations

Rare to no HIV expressing cells in tissue by in situ hybridization

Qualitative CD4 cell co-culture either negative or positive for wild-type virus (as determined by genotype)

E. Exclusion Criteria

The following criteria were used to exclude patients from the vaccination study:

Evidence of cellular immune responses to HIV-1 defined by:

Fresh CTL activity above 30% specific lysis to one or more antigens at an effector to target ratio of 25:1

CTLp above 1 in 100,000 to one or more specific HIV antigens

Pregnancy

Breast feeding

Clear evidence of HIV replication in the presence of combination drug therapy as evidence by one of the following: Plasma HIV-RNA above the level of detection on 2 consecutive tests more than 2 weeks apart, evidence of multiply-spliced (MS) HIV-RNA species in peripheral blood, or the presence of culturable virus from blood that harbors genotype consistent with drug resistance to one or more of the current antiretroviral agents included in the subject's treatment regimen.

If tissue was obtained after 24 months of therapy then patients were excluded if there was MS-HIV-RNA species demonstrated by PCR or CD4-co-culture yielded drug resistant virus (based on genotype). In addition, the presence of trapped virus in the follicular dendritic cell network as seen by in situ hybridization will resulted in exclusion.

Laboratory data:

Hemoglobin<9.0 g/l

Absolute granulocyte less than 1000 cell/mm³

Platelets less than 75,000/mm3

ALT and/or AST greater than 2.5 times the upper limit of the normal range (ULN)

Amylase above 1.5 times the ULN

Creatinine above 1.5

Bilirubin (direct) above 1.5

Allergy to eggs and/or neomycin

F. Screening Procedures

Screening was done within 60 days of receiving the first dose of vCP1452 and rgp 160.

Screening procedures included:

Complete history and physical examination

Laboratory assessments for safety at baseline:

Hematology

CBC with platelets and differential

Chemistry

Electrolytes

BUN/creatinine

Amylase

AST, ALT, alkaline phosphatase, bilirubin

Albumin, total protein

Calcium, magnesium, phosphate

Urinalysis

dipstick

microscopic analysis

Other

urine pregnancy test (prior to each vaccination)

Virology*

HIV-RNA (RT-PCR)

PBMC RT-PCR for HIV-RNA

Proviral DNA (integrated and un-integrated)

CD4+lymphocyte co-culture

Immunology*

CTLe (bulk)

CTLp

CTLe (tetramers)

HIV-specific proliferation assays to HIV antigens

HIV-specific antibody levels (p24 and gp120)

*Blood was drawn at 2 weeks, then monthly for virology and immunology. Assays other than HIV-RNA were performed at the discretion of the investigators, but no less than every three months. HIV-RNA was performed at each visit.

Pregnancy test (serum) when applicable

Virology studies

Immunology studies

G. Description of Vaccines:

ALVAC HIV (vCP1452) is a recombinant canarypox virus expressing the gag_{LAI}, protease_{LAI}, env(120)_{MN}, env(41)_{LAI}, nef, and pol genes. VCP1452 is described in U.S. Pat. Nos. 6,004,777 and 5,990,091.

vCP1452 is modified to include 2 vaccinia virus coding sequences to enhance expression in mammalian cells. The pol and nef sequences are scrambled such that no functional proteins can be expressed., Approximnately 10⁷ TCID₅₀ in 1.0 ml were given with each dose.

Recombinant gp160MN/LAI-2 is an envelope glycoprotein from HIV-1 virus expressed by vaccinia virus VV.TG.9150 on BHK₁ cells. The gp120 portion is derived from HIV_{MN} and the transmembrane gp41 portion from HIV_{LAI}. The adjuvant, PCPP, is a synthetic soluble polymer developed for its adjuvant properties. The vaccine contained 50 μg of recombinant gp160 in 500 μg PCPP (1.0 ml).

The vaccines being used in this study as well as the adjuvant are novel.

H. Schedule for vaccination

12 subjects meeting inclusion criteria were treated as follows:

ALVAC-HIV (vCP1452) and recombinant soluble gp160MN/LAI-2 were administered intramuscularly on days 0, 30, 90, 180. For ALVAC HIV (vCP1452), each vaccination dose was 1.0 mli.m. [approximately 10⁷ TCID₅₀]; for gp160 MN/LAI-2, each vaccination dose was 50 μg in 500 μg PCPP (1.0 ml).

Patients remained in the clinic area for 30 minutes after each and every vaccination. All subjects were contacted by telephone within 72 hours of each vaccination to document any adverse events. These interviews were recorded in the patient's record.

I. Patient Visits and Procedures Other than Vaccination Schedule (as above)

On day 0 subjects received:

Diary to record adverse events to be given to subjects

First dose of vaccines as outlined in protocol

On day 2 the following were performed:

Complete history and physical

Safety laboratory assessments (described above)

Urine pregnancy test (when applicable)

Virologic assessments

Immunologic assessments

3. Clarification

Screening procedures were performed within 60 days of receiving the vaccines (day 0) in addition to an additional assessment at day -2.

Week 1

Interval history and physical

Review of patient diary

Safety laboratory assessments (described above)

Week 2

Interval history and physical

Review of patient diary

Safety laboratory assessments (described above)

Virologic assessments

Immunologic assessments

Months 1-8

Interval history and physical

Review of patient diary

Safety laboratory assessments (described above)

Urine pregnancy test (when applicable)

Virologic assessments

Immunologic assessments

Post-vaccine* Interval history and physical

(1 week) Review of patient diary

Safety laboratory assessments

Virologic assessments

*within 7 days of receiving vaccine on day 0, 30, 60, 120

J. Safety Considerations

12 subjects were vaccinated and the safety and immunogenicity assessed as outlined above.

Many people have been given vaccines similar to the gp160 portion of this study without significant side effects.

The ALVAC portion given with the gp160 portion together has caused at least one of the following side effects in at least 75% of the subjects: pain and redness at the site of injection, weakness, muscle aches, joint aches, headache, and fever above 38° C. ALVAC is an avian virus (canarypox) that cannot replicate in man and therefore undergoes only one abortive cycle of replication. Over 1800 subjects received an ALVAC construct without significant serious adverse events. Additionally, over 700 subjects received ALVAC/soluble Env vaccine regimens with no severe reactions (unpublished data).

Participants were vaccinated as outpatients at the clinical site, Rockefeller University Hospital. This General Clinical research Center is staffed with highly skilled and experienced personnel. Emergency medications and equipment, known commonly as a “crash cart” were available for use in the clinic area.

Participants were monitored closely for 30 minutes post immunization for evidence of adverse events. Participants were given diaries to record any adverse event. These diaries were reviewed at each visit.

Any Grade 3 or 4 toxicity that could be definitively determined to be related to the vaccine must result in patient discontinuation.

One death has been recorded in one trial but not deemed related to the vaccine

K Immunogenicity

Antibody titers, proliferative responses and CTL activity to HIV specific antigens were measured at baseline and post-vaccination as indicated using standard techniques. Blood was drawn and cells and plasma stored for immunologic and virologic studies on days 0, 15, 30, 60, 90 120, 180, and 210 and the above assays performed. Criteria for response included: a two-fold increase in antibody titer to env and/or gag, a measurable increase in level and/or broadening of detectable fresh CTL activity and/or CTLp, and a three fold increase in proliferation index to HIV specific antigens measured in vitro.

Responders: Subjects demonstrating -an immune response to the vaccines without significant adverse events, that is, no Grade 3 or 4 nor significant local reactions, were offered the opportunity to participate in an extension that provides for vaccination every three months with identical follow-up, that is, observation in clinic for 30 minutes, telephone follow-up within 72 hours, diary cards to record temperature and adverse events, clinic visits 2 weeks after vaccination, and careful virologic monitoring, all as described above.

Failures: Subjects failing to demonstrate a response to the vaccines after day 180 as defined by the immunogenicity criteria outlined above were asked to receive vaccination with 0.5 ml tetanus toxoid to test for the ability to respond to recall antigens. Blood was drawn 1 month later to assess for an immune response (serology). Virus activity was carefully monitored with a clinic visit and virologic evaluation 2 weeks and 1 month post vaccine in addition to regularly scheduled clinic visits.

L. Extension

Individuals who are considered responders on the basis of a documented immune response, either humoral or cell mediated continued to be vaccinated with ALVAC vCP1452 and recombinant gp160 with PCPP every three months for a total of 12 months.

M. Biostatistics

Immunogenicity was determined by baseline and post vaccination measurement of: CTL activity using bulk CTL assays, CTLp frequencies, CTLe frequencies by tetramers if available, proliferation to HIV-specific antigens in vitro, and levels of HIV specific antibodies to gp120 and p24.

Subjects were followed with virologic assessments simultaneously. Increases in immunologic parameters listed above without evidence of increases in plasma HIV-RNA, PBMC-associated multiply-spliced and unspliced HIV-RNA, or abrupt changes in levels of CD4+cell-associated proviral DNA were interpreted as being the result of exposure to vaccine antigens as opposed to the result of activation of virus replication.

N. Human Subjects

1. Characterization of the Study Population

Patients over the age of 18 with documented HIV infection and treated on one of the Aaron Diamond AIDS Research Center protocols were invited to participate. All subjects met the virologic and immunologic criteria outlined above to participate. As the effect on fetuses and newborns of the vaccines used in this study, ALVAC and gp160, are unknown, all participants agreed to use double barrier contraception to prevent pregnancy.

2. Source of Research Material

After signing consent forms patients were enrolled. All antiretroviral medications were discontinued throughout the course of this study up to day 240 following initial vaccination with the ALVAC HIV (vCP1452) and gp160.

Subjects were allowed, if desired, to participate in this vaccine protocol without consenting to collection of tissue and or fluid other than blood. These were optional procedures and serve to establish the absence of virus replication as completely as possible.

3. Recruitment of Subjects

All subjects recruited for this study had been on one of the previously listed ongoing clinical trials. All participants were considered without consideration of race, sex, ethnicity, sexual orientation, or HIV risk factor. Women and members of minority groups were actively recruited to ensure representation and reflect to the best of our ability disease patterns in the local population. Patients enrolled voluntarily in this study. Decisions to either participate or not did not effect that individual's status in the ongoing studies.

4. Subject Discontinuation

Subjects, if any, experiencing a Grade 3 or 4 toxicity that could not be excluded as being due to the vaccine(s) exposure were removed from study. Subjects could withdraw at any time. This decision did not effect the ability to receive further care at the Rockefeller University Hospital.

O. Virology Plasma HIV-1 RNA levels were monitored with the Ultrasensitive RT PCR Assay (Roche) and the Bayer signal amplification assay (version 3.0) as per manufacturer's instructions.

Other details of monitoring are described above.

P. Therapy Discontinuation Post Vaccine

Of the 6 subjects completing the 180 days protocol, 4 elected to discontinue antiretroviral therapy 1 week after the last vaccine at which time HIV-1 plasma RNA levels were measured. Subjects who discontinued therapy include 1306, 1308, 1309, and 1310 and 3002. Of note, one subject had a 5^{th} vaccine injection on day 210 and discontinued therapy one week later.

Baseline characteristics at the initiation of anti-retroviral therapy are shown in Table 4:

TABLE 4

| | Days to | Log HIV-1 | CD cell | CD4/CD8 |
|---|---|---|---|---|
| Subject | treatment | RNA | count | ratio |
| 1309 | 90 | 4.2 | 500 | 0.97 |
| 1306 | 30 | 5.33 | 546 | 0.24 |
| 1308 | 7 | 6.2 | 432 | 0.92 |
| 1310 | 100 | 3.99 | 532 | 0.87 |

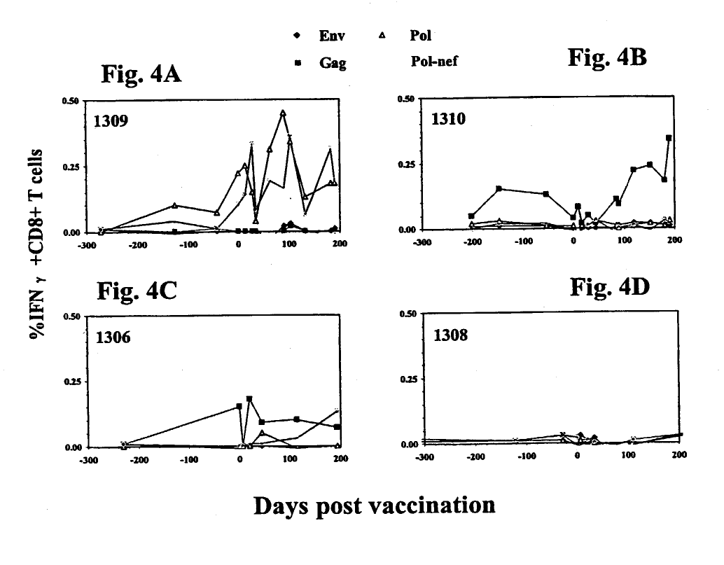

Subjects 1308 and 3002 did not respond to vaccination with an increase in the level of CD8+ IFNγ secreting cells to HIV specific antigens presented in the context of vaccinia. Subject 1310 did respond with an increase in levels of CD8+ IFNγ-secreting cells specific for Gag. Subjects 1306 and 1309 responded with an increase in CD8+ IFN-γ-secreting cells specific for more than 1 HIV-1 specific antigen (see FIG. 4). Day 0 refers to the day that subjects discontinued therapy. Period of vaccination occurred during days -217 to 0. Post-discontinuation levels of CTLe are similarly displayed.

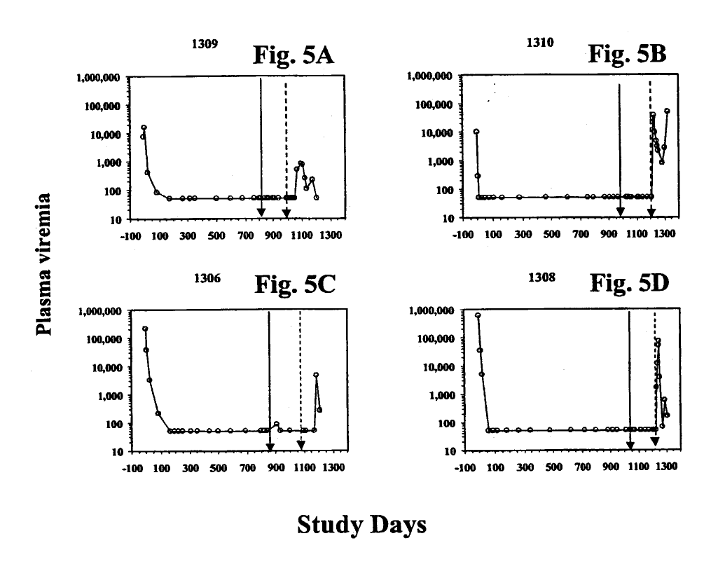

Post-therapy discontinuation subjects 1310 and 1306 rebounded after 68 and 85 days respectively. The subjects 1308 and 1310 rebounded within 23 and 13 days of therapy cessation. Furthermore the initial doubling times (t₂) of plasma viremia post therapy cessation were 4.5 and 3.2 days respectively, whereas the subjects who rebounded rapidly had a t₂ of approximately 1.5 days. The virology data for the 4 subjects are shown in FIG. 5. It is clear that Subjects 1309 and 1306 not only exhibit a delayed rebound but the mean HIV-1 RNA levels post rebound are also significantly lower than in rapidly rebounding individuals.

Post discontinuation virology data is shown in Table 6:

TABLE 6

| | Days to | | Time to | | |
|---|---|---|---|---|---|
| | detectable | Log peak | peak | Current log | Days off |
| Subject | HIV-1 RNA | viremia | viremia | HIV-1 RNA | therapy |
| 1310 | 68 | 2.93 | 95 | 2.91 | 239 |
| 1306 | 85 | 3.73 | 145 | 3.73 | 145 |
| 1308 | 23 | 4.15 | 77 | 3.55 | 132 |
| 1310 | 13 | 4.95 | 105 | 4.77 | 135 |

Safety of vCP1452

| | |
|---|---|
| Grade 3 or 4 toxicities | 0/8 |
| Significant systemic toxicities | 0/8 |
| Local tenderness | 8/8 |
| Swelling, redness or induration at the site of vaccination | 0/8 |
| Evidence of activation of virus replication | 0/8 |
| Worsening of baseline adverse events associated with chronic | 0/8 |
| antiretroviral therapy | |

References

1. Ho D D, Sargadharan M G, Resnick L, DiMarzo-Veronese F, Rota T R, Hirsch M S. Primary human T-lymphotropic virus type III infection. Ann. Int. Med. 1985; 103:880-883.

2. Daar E S, Moudgil T, Meyer R D, Ho D D. Transient high levels of viremia in patients with primary human immunodeficiency virus type 1 infection. N. Engl. J. Med. 1991; 324:961-964.

3. O'Brien T R, Blattner W A, Waters D, et al. Serum HIV-1 RNA levels and time to development of AIDS in the multicenter hemophilia cohort study. J. Amer. Med. Assoc. 1996; 276:105-110.

4. Koup R A, Safrit J T, Cao Y, et al. Temporal association of cellular immune responses with the initial control of viremia in primary HIV-1 syndrome. J. Virol. 1994; 68:4650-4655.

5. Moore J P, Cao Y, Ho D D, Koup R A. Development of the anti-gp120 antibody response during seroconversion to human immunodeficiency virus type 1. J. Virol. 1994; 68:5142-5155.

6. Fauci A S. The human immunodeficiency virus: infectivity and mechanisms of pathogenesis. Science 1988; 239:617-622.

7. Pantaleo G, Graziosi C, Demarest J F, et al. HIV infection is active and progressive in lymphoid tissue during the clinically latent stage of disease. Nature 1993; 362:355-358.

8. Embretson J, Zupacic M, Ribas J L, et al. Massive covert infection of helper T lymphocytes and macrophages by HIV during the incubation period of AIDS. Nature 1993; 362:359-362.

9. Ho D D, Neumann A U, Perelson A S, Chen W, Leonard J M, Markowitz M. Rapid turnover of plasma virions and CD4 lymphocytes in HIV-1 infection. Nature 1995; 373:123-126.

10. Mellors J W, Rinaldo Jr. C R, Gupta P, White R M, Todd J A, Kingsley L A. Prognosis in HIV-1 infection predicted by the quantity of virus in plasma Science 1996; 272:1167-1170.

11. DeBouck C. The HIV-1 protease as a therapeutic target for AIDS. AIDS Res. Hum. Retroviruses 1992; 8:153-164.

12. Kohl N E, Emini E A, Schleif W A, et al. Active human immunodeficiency virus protease is required for viral infectivity. Proc. Natl. Acad. Sci. U.S.A. 1988; 85:4686-4690.

13. Roberts N A, Martin J A, Kinchington D, et al. Rational design of peptide-based HIV proteinase inhibitors. Science 1990; 248:358-361.

14. Kempf D, Marsh K, Denissen J, al. e. ABT-538 is a potent inhibitor of human immunodeficiency virus protease and has high oral bioavailability in humans. Proc. Natl. Acad. Sci. USA 1995; 92:2484-2488.

15. Patick A K, Mo H, Markowitz M, et al. Antiviral and resistance studies of AG1343, an orally bioavailable inhibitor of human immunodeficiency virus protease. Antimicrobial Agents & Chemo. 1996; 40:292-297.

16. Vacca J P, Dorsey B D, Schlief W A, et al. L-735,524 ; An orally bioavailable human immunodeficiency virus type 1 protease inhibitor. Proc. Natl. Acad. Sci. USA 1994; 91:4096-4100.

17. Hammer S, Squires K, Hughes M, et al. A Controlled trial of Two Nucleoside Analogues plus Indinavir in Persons with HIV Infecion and CD4 Cell Counts of 200 Per Cubic Millimeter or Less. N Engl J Med 1997; 337:725-733.

18. Gulick R, Mellors J, Havlir D, et al. Treatment with Indinavir, ZDV, and Lamivudine in Adults with HIV Infection and Prior Antiretroviral Therapy. N Engl J Med 1997; 337:734-739.

19. Cohen C, Sun E, Cameron W, et al. Ritonavir-saquinavir combination treatment in HIV-infected patients. ICAAC, New Orleans, La. 1996; LB7b.

20. Perelson A S, Neumann A U, Markowitz M, Leonard J M, Ho D D. HIV-1 dynamics in vivo: virion clearance rate, infected cell life span, and viral generation time. Science 1996; 271:1582-1586.

21. Perelson A, Essunger P, Cao Y, et al. Decay Characteristics of HIV-1 Infected Compartments During Combination Therapy. Nature 1997; 387:188-190.

22. Chun T-W, Carruth L., Finzi D. S X, DiGiuseppe J., Taylor H., Hermankova M.,Chadwick K., Margolick J., Quinn T., Kuo Y.,Brookmeyer R., Zeiger M., Barditch-Crovo P., Siliciano R. Quantification of latent tissue reservoirs and total body viral load in HIV-1 infection. Nature 1997; 387:183-188.

23. Chun T W, Finzi D, Margolick J, Chadwick K, Schwartz D, Siliciano R F. In vivo fate of HIV-1-infected T cells: quantitative analysis of the transition to stable latency. Nat. Med. 1995; 1:1284-1290.

24. Vesanen M, Cao Y, Hurley A, Schluger R, Ho D, M M. HIV-1 proviral DNA decay rate in patients treated with potent antiretroviral regimens, International Workshop on HIV Drug Resistance, Treatment Strategies and Eradication, St Petersburg, Fla., 1997.

25. Markowitz M, Cao Y, Hurley A, et al. Triple therapy with AZT and 3TC in combination with nelfinavir mesylate in 12 antiretroviral-naive subjects chronically infected with HIV-1. XI International Conference on AIDS, Vancouver, British Columbia, Canada 1996; Supplement:LB.B. 6031.

26. Markowitz M, Y. C, Hurley A, et al. Triple therapy with AZT,3TC, and ritonavir in 12 subjects newly infected with HIV-1, XI International Conference on AIDS, Vancouver, Canada, 1996.

27. Markowitz M, Cao Y, Vesanen M, et al. Recent HIV infection treated with AZT, 3TC, and a potent protease inhibitor., 4th Conference on Retroviruses and Opportunistic Infections, Washington, D.C., 1997.

28. Zhu T, Mo H, Wang N, et al. Genotypic and phenotypic characterization of HIV-1 in patients with primary infection. Science 1993; 261:1179-1181.

29. Zhu T, Wang N, Carr A, et al. Genetic characterization of human immunodeficiency virus type 1 in blood and genital secretions: evidence for viral compartmentalization and selection during sexual transmission. J. Virol. 1996; 70:3098-3107.

30. Haynes B, Pantaleo G, Fauci A S. Toward an understanding of the correlates of protective immunity to HIV infection. Science 1996; 271:324327.

31. Pantaleo G, Menzo S, Vaccarezza M, et al. Studies in subjects with long-term nonprogressive human immunodeficiency virus infection. N. Eng. J. Med 1995; 332.

32. Cao Y, Qin L, Zhang L, Safrit J, Ho D D. Virologic and immunologic characterization of long-term survivors of human immunodeficiency virus type 1 infection. New Eng. J. Med. 1995; 332:201-208.

33. Pantaleo G, Demarest JF, Schacker T, et al. The qualitative nature of the primary immune response to HIV infection is a prognosticator of disease progression independent of the inital level of plasma viremia. Proc Natl Acad Sci 1997; 94:254258.

34. Pantaleo G, Demarest J F, Soudeyns H, et al. Major expansion of CD8+ T lymphocytes with a predominant Vβ usage during the primary immune response to HIV. Nature 1994; 370:463467.

35. Cao Y, Qing L, Zhang L Q, Safrit J T, Ho D D. Virological and immunological characterization of long-term survivors of HIV-1 infection. N. Engl. J. Med. 1994; 332:201-208.

36. Moore J P, Cao Y, Qing L, et al. Primary isolates of human immunodeficiency virus type 2 are relatively resistant to neutralization by monoclonal antibodies to gp120. J. Virol. 1995; 69:101-109.

37. Excler. J, Plotkin S. The prime-boost concept applied to HIV preventive vaccines. AIDS 1997; 11:S127-S137.

38. Berzofsky J A, Bensussan A, Cease K B, et al. Antigenic peptides recognized bt T lymphocytes from AIDS viral envelope-immune humans. Nature 1988; 334:706-708.

39. Redfield R R, Wright D C, James W D, Jones T S, Brown C, Burke D S. Disseminated vaccinia in a military recruit with HTLV-III disease. New Engl. J. Med. 1987; 316:673-676.

40. Graham B S, Matthews T J, Belshe R B, et al. Augmentation of human immunodeficiency virus type 1 neutralizing antibody by priming with gp160 recombinant vaccinia and boosting with rgp 160 in vaccinia-naive adults. J. Infect. Dis. 1993; 167:533-537.

41. Pialoux G, Exder J-L, Riviere Y, et al. A prime boost approach to HIV preventitive vaccine using a recombinant canarypox virus expressing glycoprotein 160 (MN) followed by a recombinant glycorpotein 160 (MN/LAI). AIDS Res. Hum.

Retroviruses 1995; 11:373-382.

42. Fleury B, Janvier G, Pialoux G, et al. Memory cytotoxic T lymphocyte responses in human immunodeficiency virus type 1 (HIV-1)-negative volunteers immunized with a recombinant canarypox expressing gp160 of HIV-1 and boosted with a recombinant gp160. J. Inf. Dis. 1996; 174:73-4738.

43. Mulder J, McKinney N, Christopherson C, Sninsky J, Greenfield L, Kwok S. Rapid and simple PCR assay for quantification of HIV-1 RNA in plasma: Application to acute retroviral infection. Journal of Clinical Microbiology 1994; 32:292-300.

44. Saksela K, Stevens C, Rubinstein P, Baltimore D. Human immunodeficiency virus type 1 mRNA expression in peripheral blood cells predicts disease progression independently of the numbers of CD4+ lymphocytes. Proc. Natl. Acad. Sci. USA 1994; 91:1104-1108.

45. Saksela K, Stevens C E, Rubinstein P, Taylor P E, Baltimore D. HIV-1 messenger RNA in peripheral blood mononuclear cells as an early marker of risk for progression to AIDS. Ann. Intern. n Med. 1995; 123:641-648.

46. Vesanen M, Markowitz M, Cao Y, Ho D D, Saksela K. HIV-1 mRNA plicing pattern in infected persons is determined by the proportion of newly infected cells. Virology 1997; 236:104-109.

47. Embretson J, Zupacic M, Ribas J L, et al. Massive covert infection of helper T lymphocytes and macrophages by HIV during the incubation period of AIDS. Nature 1993; 362:359-362.

48. Fox C, Tenner-Racz K, Racz P, Firpo A, Pizzo P, Fauci A. Lymphoid Germinal Centers Are Reservoirs of Human Immunodeficiency Virus Type 1 RNA. Jounal of Infectious Diseases 1991; 164:1051-1057.

49. Nixon D F, Townsend ARM, Elvin J G, Rizza C R, Gallwey J, McMichael A J. HIVV-1 gag-specific cytotoxic T lymphocytes defined with recombinant vaccinia virus and synthetic peptides. Nature 1988; 336:484-487.

We claim:

1. A method of treating HIV-infected patients, the method comprising:
a) subjecting the patient to antiviral therapy;
b) administering to the subject one or a plurality of nucleic acid-based vaccines that enter the patient's cells and intracellularly produce one or a plurality of HIV-specific immunogens for presentation on the cell's MHC class I and MHC class II molecules in an amount sufficient to stimulate an HIV-specific CD8+ and CD4+ responses;
c) ceasing said antiviral therapy.

2. The method according to claim 1, wherein the antiviral therapy is HAART.

3. The method according to claim 2 wherein before administering the nucleic acid-based vaccine or vaccines, the patient has a viral load of less than 10,000 viral copies per ml of plasma and a CD4+ T-cell count of above 300 cells/ml before administration of vaccine.

4. The method according to claim 2 wherein before administering the nucleic acid-based vaccine or vaccines, the patient has a viral load of less than 5,000 viral copies per ml of plasma CD4+ T-cell count of above 300 cells/ml before administration of vaccine.

5. The method according to claim 2 wherein before administering the nucleic acid-based vaccine or vaccines, the patient has a viral load of less than 1,000 viral copies per ml of plasma CD4+ T-cell count of above 300 cells/ml before administration of vaccine.

6. The method according to claim 2 wherein before administering the nucleic acid-based vaccine or vaccines, the patient has a viral load of less than 10,000 viral copies per ml of plasma CD4+ T-cell count of above 500 cells/ml before administration of vaccine.

7. The method according to claim 2 wherein before administering the nucleic acid-based vaccine or vaccines, the patient has a viral load of less than 5,000 viral copies per ml of plasma CD4+ T-cell count of above 500 cells/ml before administration of vaccine.

8. The method according to claim 2 wherein before administering the nucleic acid-based vaccine or vaccines, the patient has a viral load of less than 1,000 viral copies per ml of plasma CD4+ T-cell count of above 500 cells/ml before administration of vaccine.

9. The method according to claim 2 wherein the patient exhibits CD4+ and/or CD8+ T-cell responses to HIV.

10. The method according to claim 2 wherein the patient exhibits CD4+ and CD8+ T-cell responses to envelope epitopes.

11. The method according to claim 2 wherein the patient exhibits CD4+and CD8+ T cell responses to Gag epitopes.

12. The method according to claim 2 wherein the patient has lost his CD4+ and/or CD8+ T cell responses to HIV antigens.

13. The method according to claim 2 wherein the patient has lost his CD4+ and CD8+ T cell responses to envelope and Gag HIV epitopes.

14. The method according to claim 2 wherein the HIV specific immunogen is gp120.

15. The method according to claim 2 wherein the HIV-specific immunogen is Gag.

16. The method according to claim 2 wherein the nucleic acid-based vaccine comprises one or a plurality of naked DNAs encoding one or a plurality of HIV-specific immunogens.

17. The method according to claim 2 wherein the nucleic acid-based vaccine comprises one or a plurality of DNA vectors encoding one or a plurality of HIV-specific immunogens.

18. The method according to claim 17 wherein the DNA vector is a recombinant virus.

19. The method according to claim 17 wherein the DNA vector is a recombinant attenuated virus.

20. The method according to claim 18 wherein the recombinant attenuated virus is selected from the group consisting of adenoviruses, adeno-associated viruses, human influenza viruses, herpes simplex virus (HSV), coksackie viruses, Vesicular stomatitis viruses (VSV), and alphaviruses.

21. The method according to claim 18 wherein the recombinant attenuated virus is a poxvirus.

22. The method according to claim 21 wherein the recombinant attenuated virus is selected from the group consisting of vaccinia, avipox, fowlpox, and canarypox.

23. The method according to claim 22 wherein the recombinant attenuated virus is NYVAC or ALVAC.

24. The method according to claim 18 wherein the recombinant attenuated virus is MVA.

25. The method according to claim 18 wherein the HIV-specific immunogen is a structural protein.

26. The method according to claim 25 wherein the HIV-specific immunogen is a structural protein selected from the group consisting of gp 160, gp 120, gp 41, and Gag.

27. The method according to claim 18 wherein the HIV-specific immunogen is a non-structural protein.

28. The method according to claim 27 wherein the HIV-specific immunogen is a non-structural protein encoded by a gene selected from the group consisting of rev, tat, nef, vif, and vpr.

29. The method according to claim 18 wherein the HIV-specific Immunogen is selected from the group consisting of HIV-1 Gag, gp120, NefCTL, PoICTL epitopes.

30. The method according to claim 18 wherein the HIV-specific immunogen presents at least one epitope selected from the group consisting of ELDKWA, LDKW, Nef1, Nef2, the V3 loop, Pol1, Pol2 and Pol3.

31. The method according to claim 18 wherein the HIV-specific immunogen presents at least one epitope of a peptide selected from the group consisting of gp 160, gp 120, gp 41, Gag, and at least one protein encoded by the rev, tat, nef, vif, or vpr gene.

32. The method according to claim 2 wherein the nucleic acid-based vaccine comprises a construct selected from the group consisting of vCP1452, vCP1433, vCPI25, vCP205, and VCP300.

33. The method according to any one of claims 18, 21, 23, 26, 28, and 31 wherein the vaccine is administered. simultaneously or sequentially, with a soluble HIV antigen.

34. The method according to claim 33, wherein the soluble HIV antigen is gp160.

35. The method according to claim 33, wherein the soluble HIV antigen is recombinant gp 160MN/LAI.
